(19) United States
(12) Patent Application Publication
(10) Pub. No.: US 2023/0251275 A1
(43) Pub. Date: Aug. 10, 2023

(54) NPERSEVERE: BIOMARKERS ESTIMATING BASELINE MORTALITY RISK FOR NEONATAL SEPSIS AND NECROTIZING ENTEROCOLITIS

(71) Applicant: Children's Hospital Medical Center, Cincinnati, OH (US)

(72) Inventors: Faris N. Al Gharaibeh, Cincinnati, OH (US); Hector R. Wong, Cincinnati, OH (US)

(21) Appl. No.: 18/104,055

(22) Filed: Jan. 31, 2023

Related U.S. Application Data
(60) Provisional application No. 63/305,613, filed on Feb. 1, 2022.
(60) Provisional application No. 63/350,531, filed on Jun. 9, 2022.
(60) Provisional application No. 63/404,778, filed on Sep. 8, 2022.

(51) Int. Cl. G01N 33/68

(52) U.S. Cl. CPC G01N 33/6893; G01N 33/6869; G01N 2800/26; G01N 2800/56; G01N 2800/52

(57) ABSTRACT

Methods and compositions disclosed herein generally relate to methods of identifying, validating, and measuring clinically relevant, quantifiable biomarkers of diagnostic and therapeutic responses for blood, vascular, cardiac, and respiratory tract dysfunction, particularly as those responses relate to sepsis and/or necrotizing enterocolitis in pediatric patients. In particular, the invention relates to identifying two or more biomarkers associated with septic shock in pediatric patients, obtaining a sample from a pediatric patient having at least one indication of sepsis and/or necrotizing enterocolitis, then quantifying from the sample an amount of two or more of said biomarkers, wherein the level of said biomarker correlates with a predicted outcome.

### CROSS REFERENCE TO RELATED APPLICATION

The present application claims the benefit of priority under 35 U.S.C. § 119(e) to U.S. Provisional Application No. 63/305,613, BIOMARKERS TO ESTIMATE BASELINE RISK OF MORTALITY IN NEONATES WITH SEPSIS AND NECROTIZING ENTEROCOLITIS, filed on Feb. 1, 2022; U.S. Provisional Application No. 63/350,531, NPERSEVERE: BIOMARKERS ESTIMATING BASELINE MORTALITY RISK FOR NEONATAL SEPSIS AND NECROTIZING ENTEROCOLITIS, filed on Jun. 9, 2022; and U.S. Provisional Application No. 63/404,778, BIOMARKERS ESTIMATING BASELINE MORTALITY RISK FOR NEONATAL SEPSIS, filed on Sep. 8, 2022, which are currently co-pending herewith and which is incorporated by reference in its entirety.

### STATEMENT REGARDING FEDERALLY-SPONSORED RESEARCH

This invention was made with government support under Grant No. R35GM126943 awarded by the National Institutes of Health. The government has certain rights in the invention.

### FIELD OF THE INVENTION

The invention disclosed herein generally relates to the identification and validation of clinically relevant, quantifiable biomarkers of diagnostic and therapeutic responses for blood, vascular, cardiac, and respiratory tract dysfunction, in particular biomarkers of septic shock.

### BACKGROUND

Sepsis remains a major cause of mortality and morbidity in neonates, with the very low birth weight population being the most vulnerable [1,2]. Biologically plausible interventions failed to show impact on mortality from neonatal sepsis [3,4], which could be explained in part by heterogeneity of neonatal sepsis and unequal baseline mortality risk in study arms.

For decades, biomarker research in sepsis has been heavily focused on identifying culture-positive sepsis. Biomarkers can also have utility for estimation of baseline mortality risk, which is fundamental to clinical practice and research. Prognostic and predictive biomarker research is lacking in the newborn intensive care unit, particularly for prognostic biomarker research for neonatal sepsis and necrotizing enterocolitis. Biomarker research could help in prognostication in neonatal sepsis, yet studies are heavily skewed towards early diagnosis of culture-positive sepsis. Biomarkers such as CRP [5], IL-6 [6], IL-8 [7], and IL-10 [8] have been tested in various settings for this purpose, but study findings have not changed current clinical practice.

### SUMMARY OF THE INVENTION

Embodiments of the invention relate to methods of classifying a neonate patient with sepsis and/or necrotizing enterocolitis as high risk of mortality and/or complicated course, or other than high risk of mortality and/or complicated course, the method including: obtaining a sample from a neonate patient with sepsis and/or necrotizing enterocolitis at a first time point; analyzing the sample to determine gene expression or serum protein biomarker concentrations of one or more biomarkers including IL-8; determining whether the gene expression or serum levels of each of the biomarkers are greater than a respective cut-off level; and classifying the patient as high risk of mortality and/or complicated course, or other than high risk of mortality and/or complicated course, based on the determination of whether the levels of each of the biomarkers are greater than the respective cut-off level.

In some embodiments of the methods, a classification of other than high risk of mortality and/or complicated course comprises a non-elevated level of IL-8.

Some embodiments further include analyzing the sample to determine serum protein biomarker concentrations of one or more additional biomarkers including MMP8, and determining platelet count of the neonate patient, where a classification of high risk of mortality and/or complicated course includes: a) an elevated serum level of IL-8, and a non-elevated median platelet count per mm³; or b) an elevated serum level of IL-8, an elevated median platelet count per mm³, and an elevated serum level of MMP8; and where a classification of other than high risk of mortality and/or complicated course includes: c) a non-elevated serum level of IL-8; or d) an elevated serum level of IL-8, an elevated median platelet count per mm³, and a non-elevated serum level of MMP8.

In some embodiments of the methods, a classification of high risk of mortality and/or complicated course can include: a) a highly elevated level of IL-8; or b) an elevated level of IL-8, and an elevated level of CCL3; and wherein a classification of other than high risk of mortality and/or complicated course can include: c) a non-highly elevated level of IL-8, and a non-elevated level of CCL3; or d) a non-elevated level of IL-8, and an elevated level of CCL3.

In some embodiments, the serum biomarker levels can be determined by quantification of serum protein biomarker concentrations. In some embodiments, the serum biomarker levels can be determined by concentrations and/or by cycle threshold (CT) values. In some embodiments, the gene expression values (mRNA expression levels) can be determined by arbitrary units of mRNA counts. In some embodiments, mRNA counts can be generated by the NanoString nCounter platform (NanoString Technologies, Seattle, Wash.). In some embodiments, mRNA counts can be normalized to one or more housekeeping genes, such as, for example, four housekeeping genes (e.g. β-2-microglobulin (B2M), folylpolyglutamate synthase (FPGS), 2,4-dienoyl CoA reductase 1 (DECR1), and peptidylprolyl isomerase B (PPIB)), as has been described previously (Wong 2015). In some embodiments, expression values can be normalized to the geometric mean of the housekeeping genes.

In some embodiments, the serum biomarker levels can be determined by serum protein biomarker concentration, and the median platelet count can be determined by counting the median number of platelets per mm³, where: a) an elevated level of IL-8 corresponds to a serum IL-8 concentration greater than 297 pg/mL; b) an elevated median platelet count per mm³ corresponds to a median platelet count per mm³ greater than 127,000 per mm³; and c) an elevated level of MMP8 corresponds to a serum MMP8 concentration greater than 111,846 pg/mL.

In some embodiments, the serum biomarker levels can be determined by serum protein biomarker concentration, where: a) an elevated level of IL-8 corresponds to a serum IL-8 concentration greater than 297 pg/mL; b) a highly elevated level of IL-8 corresponds to a serum IL-8 concentration greater than 7465 pg/mL; and c) an elevated level of CCL3 corresponds to a serum CCL3 concentration greater than 47 pg/mL.

In some embodiments, an elevated level of IL-8 corresponds to a serum IL-8 concentration greater than 297 pg/mL.

In some embodiments, the determination of whether the levels of the two or more serum biomarkers are non-elevated above a cut-off level includes applying the biomarker expression level data to a decision tree including the two or more biomarkers. In some embodiments, the biomarker expression level data can be applied to the decision tree of FIG. 7. In some embodiments, the biomarker expression level data can be applied to the decision tree of FIG. 15.

In some embodiments, the classification of other than high risk of mortality and/or complicated course includes a classification of low risk of mortality and/or complicated course. In some embodiments, the complicated course can include persistence of two or more organ dysfunctions on day 7 of illness and/or vasopressor use. In some embodiments, the complicated course can include cardiovascular, respiratory, renal, hepatic, hematologic, and/or neurologic dysfunction. In some embodiments, the complicated course can include cardiovascular dysfunction. In some embodiments, the complicated course can include vasopressor use, and/or dysfunction in one or more organs selected from heart, lungs, kidneys, liver, blood, and brain.

In some embodiments, the high risk of mortality and/or complicated course by day 7 of the sepsis and/or necrotizing enterocolitis, or the other than high risk of mortality and/or complicated course by day 7 of sepsis and/or necrotizing enterocolitis, can be determined.

In some embodiments, the classification can be combined with one or more patient demographic data and/or clinical characteristics and/or results from other tests or indicia of sepsis and/or necrotizing enterocolitis and/or one or more additional biomarkers. In some embodiments, the one or more additional biomarkers is selected from wherein the biomarkers can further include one or more selected from the group consisting of Heat shock protein 70 kDA (HSP70), HSPA1b (Heatshock Protein A1b), GZMB (Granzyme B), Interleukin-1 α (IL-1a), MMP-8 (Matrix Metallopeptidase 8), and platelet count. In some embodiments, the demographic data and/or clinical characteristics and/or results from other tests or indicia of sepsis and/or necrotizing enterocolitis can include at least one selected from the sepsis and/or necrotizing enterocolitis causative organism, the presence or absence or chronic disease, and/or the chronological age, gestational age at birth, birth weight, gender, race, and/or co-morbidities of the patient. In some embodiments, the patient demographic data can include the chronological age, gestational age at birth, and/or birth weight of the patient. In some embodiments, the patient demographic data can include the chronological age or the gestational age at birth of the patient.

In some embodiments, the classification can be combined with one or more additional population-based risk scores.

In some embodiments, the sample can be obtained within the first hour of presentation with sepsis and/or necrotizing enterocolitis. In some embodiments, the sample can be obtained within the first 24 hours of presentation with sepsis and/or necrotizing enterocolitis.

Some embodiments of the methods further include administering a treatment including one or more high risk therapy to a neonate patient that is classified as high risk, or administering a treatment excluding a high risk therapy to a neonate patient that is not high risk, or to provide a method of treating a neonate patient with sepsis and/or necrotizing enterocolitis.

In some embodiments, the one or more high risk therapy includes at least one selected from immune enhancing and/or modulating therapy, extracorporeal membrane oxygenation/life support, plasmapheresis, pulmonary artery catheterization, and/or high-volume continuous hemofiltration. In some embodiments, the immune enhancing and/or modulating therapy includes administration of GMCSF, IVIG, anit-IL-8, anti-IL-2, interleukin-7, and/or anti-PD-1.

In some embodiments, the patient classified as high risk of mortality and/or complicated course is enrolled in a clinical trial. In some embodiments, a treatment including one or more therapy excluding a corticosteroid can be administered to the patient in the clinical trial. In some embodiments, an outcome can be improved in the patient with sepsis and/or necrotizing enterocolitis.

Some embodiments of the methods further include: obtaining a second sample from the treated patient at a second time point; analyzing the second sample to determine the expression levels of expression levels of one or more biomarkers including IL-8; determining whether the protein biomarker expression levels of each of the biomarkers are greater than a respective cut-off protein biomarker expression level; and classifying the patient as high risk of mortality and/or complicated course, or other than high risk of mortality and/or complicated course, based on the determination of whether the expression levels of each of the biomarkers are greater than the respective cut-off expression level; maintaining the treatment being administered if the patient's high risk classification has not changed, or changing the treatment being administered if the patient's high risk classification has changed. In some embodiments, the method further includes determining biomarker expression levels of MMP8 and/or CCL3, and/or measuring platelet count.

In some embodiments, the second time point can be at least 18 hours after the first time point. In some embodiments, the second time point is in the range of 24 to 96 hours, or longer, after the first time point. In some embodiments, the second time point can be about 1 day, 2 days, 3 days, or longer, after the first time point. In some embodiments, the second time point can be about 2 days after the first time point. In some embodiments, the first time point can be at day 1, wherein day 1 is within 24 hours of a sepsis and/or necrotizing enterocolitis diagnosis, and the second time point can be at day 3. In some embodiments, the patient classified as high risk after the second time point can be administered one or more high risk therapy. In some embodiments, the patient classified as high risk and administered one or more high risk therapy after the first time point is not classified as high risk after the second time point.

In some embodiments, the patient can be less than five weeks old. In some embodiments, the patient can be less than one year of age and admitted to the newborn intensive care unit. In some embodiments, the patient can have necrotizing enterocoloitis. In some embodiments, the patient can have sepsis and necrotizing enterocoloitis.

Some embodiments of the invention further include diagnostic kits, tests, or arrays including a reporter hybridization probe, and a capture hybridization probe specific for each of two or more mRNA, DNA, and/or protein biomarkers including IL-8 and further including MMP8 and/or CCL3. Some embodiments further include a collection cartridge for immobilization of the hybridization probes. In some embodiments, the reporter and the capture hybridization probes include signal and barcode elements, respectively.

Some embodiments of the invention further include apparatuses or processing devices suitable for detecting two or more biomarkers including IL-8 and further including MMP8 and/or CCL3.

Some embodiments of the invention further include compositions or reaction mixtures, including a reporter hybridization probe, and a capture hybridization probe specific for each of two or more mRNA, DNA, and/or protein markers including IL-8 and further including MMP8 and/or CCL3.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1 depicts the study flow chart for Cohort 1. 86 events were evaluated, after exclusions, 71 neonates were included in the analysis.

FIG. 2 depicts the PERSEVERE II classification tree. This is the PERSEVERE II tree which was used to test its ability in Cohort 1. Neonates who were classified in terminal nodes (TN) labeled TN1, TN2, TN5, and TN8 are considered low-risk and those classified in terminal nodes labeled TN3, TN4, TN6, TN7, and TN9 are considered high-risk.

FIG. 3 demonstrates that IL-8 is a candidate biomarker for mortality prediction in Cohort 1. FIG. 3A. The median and the minimum to maximum range of IL-8 levels in non-survivors vs. survivors, 1486 pg/mL vs. 219 pg/mL respectively, p 0.0001 using Mann-Whitney U test. FIG. 3B. Receiving-operator curve for IL-8, area under the curve 0.83 (95% CI 0.72 to 0.94).

FIG. 4 demonstrates that CCL3 is higher in non-survivors compared to survivors in Cohort 1. FIG. 4A. The median and the minimum and maximum range of CCL3 levels in non-survivors vs. survivors, 81 pg/mL vs. 45 pg/mL respectively, p 0.009 using Mann-Whitney test. FIG. 4B. Receiving-operator curve for CCL3, area under the curve 0.73 (95% CI 0.58 to 0.87).

FIG. 5 demonstrates that HSP A1b Is Higher in Non-Survivors Compared to Survivors in Cohort 1. FIG. 5A. The median and the minimum and maximum range of HSP A1b levels in non-survivors vs. survivors, 600765 pg/mL vs. 436901 pg/mL respectively, p 0.03 using Mann-Whitney test. FIG. 5B. Receiving-operator curve for HSP A1b, area under the curve 0.69 (95% CI 0.55 to 0.83).

FIG. 6 depicts the nPERSEVERE-1 classification tree. This decision tree was made by using all the available PERSEVERE serum biomarkers, gestational age, and birthweight. The algorithm was instructed to stop branching once a node has 5% of the original cohort. Terminal nodes labeled TN1, TN2, TN3, TN5 are predicted survivors, while those labeled TN4 and TN6 are predicted non-survivors.

FIG. 7 depicts the pruned final version of nPERSEVERE-1. After pruning the gestational age branching, this tree was used for testing in Cohort 1. Terminal nodes that are labeled TN1 and TN2 are predicted survivors while those labeled TN3 and TN4 are predicted non-survivors. The survival rate of TN1 and TN2 is 98% compared to 54% of those classified as TN3 and TN4.

FIG. 8 depicts the survival curves of high- and low-risk patients in Cohort 1. Patients classified as high-risk according to nPERSEVERE-1 (nHR; lower circles, continuous line) had a 53.8% survival rate compared to 97.8% in the low-risk group (nLR; upper circles, continuous line). Patients classified as high-risk according to PERSEVERE II (pHR; lower squares, interrupted line) had a 70.5% survival rate compared to 91.9% in the low-risk group (pLR; upper squares, interrupted line).

FIG. 9 depicts the study flow chart for Cohort 2. 71 events were evaluated, after exclusions, 58 neonates were included in the analysis.

FIG. 10 demonstrates that IL-8 is a biomarker for mortality prediction. FIG. 10A. The median and the minimum to maximum range of IL-8 levels in non-survivors (left) vs. survivors (right), 10,114 pg/mL vs. 207 pg/mL respectively, p 0.0001 using the Mann-Whitney U test. FIG. 10B. Receiving-operator curve for IL-8, area under the curve 0.87 (95% CI 0.77 to 0.98).

FIG. 11 demonstrates that platelet count is lower in non-survivors compared to survivors in Cohort 2. FIG. 11A. The median and the minimum and maximum range of platelet counts in non-survivors (left) vs. survivors (right), 104 000 per mm³ vs. 246 000 per mm³ respectively, p 0.006 using Mann-Whitney test. FIG. 11B. Receiving-operator curve for platelet count, area under the curve 0.78 (95% CI 0.60 to 0.97).

FIG. 12 demonstrates that CCL3 is higher in non-survivors compared to survivors in Cohort 2. FIG. 12A. The median and the minimum and maximum range of CCL3 levels in non-survivors (left) vs. survivors (right), 104 pg/mL vs. 39 pg/mL respectively, p 0.03 using Mann-Whitney test. FIG. 12B. Receiving-operator curve for CCL3, area under the curve 0.73 (95% CI 0.55 to 0.91).

FIG. 13 demonstrates that HSP A1b is higher in non-survivors compared to survivors in Cohort 2. FIG. 13A. The median and the minimum and maximum range of HSP A1b levels in non-survivors (left) vs. survivors (right), 1 390 000 pg/mL vs. 433 874 pg/mL respectively, p 0.03 using Mann-Whitney test. FIG. 13B. Receiving-operator curve for HSP A1b, area under the curve 0.75 (95% CI 0.60 to 0.91).

FIG. 14 depicts the nPERSEVERE-2 classification tree prior to pruning. This decision tree was made by using all the available PERSEVERE serum biomarkers, gestational age, and birthweight. The algorithm was instructed to stop branching once a node has 5% of the original cohort. Terminal nodes labeled TN2, TN3, TNS, and TN7 are predicted survivors, while those labeled TN1, TN4, and TN6 are predicted non-survivors. this iteration is 78% sensitive and 92% specific for mortality, p<0.0001. Upon generating the ROC curve, the calculated AUC for this model is 0.96 (95% CI 0.91-0.99), p<0.0001.

FIG. 15 depicts the pruned final version of nPERSEVERE-2. After pruning, this tree was chosen to test in Cohort 2. The terminal nodes that are labeled TN1 and TN3 are low risk (predicted survivors), while those labeled TN2 and TN4 are high risk (predicted non-survivors). The survival rate of those classified in the low risk nodes is 100%, compared to 44% of those classified in high risk nodes.

FIG. 16 depicts survival trends of neonates in Cohort 2 according to nPERSEVERE-2 classification. FIG. 16A. Neonates in Cohort 2 classified as high risk (nHR red squares, bottom line) had a survival rate of 44% compared to 100% survival for neonates classified as low risk (nLR green squares, top line), p<0.0001. The entire cohort's (EC black squares, middle line) survival curve is depicted for comparison. FIG. 16B. Distribution of mortality timing shows that most deaths occur within the first month of the sepsis event (67%), with the majority occurring in the first two weeks.

### DETAILED DESCRIPTION OF THE INVENTION

Unless otherwise noted, terms are to be understood according to conventional usage by those of ordinary skill in the relevant art.

All references cited herein are incorporated by reference in their entirety. Also incorporated herein by reference in their entirety include: U.S. Patent Application No. 61/595,996, BIOMARKERS OF SEPTIC SHOCK, filed on Feb. 7, 2012; U.S. Provisional Application No. 61/721,705, A MULTI-BIOMARKER-BASED OUTCOME RISK STRATIFICATION MODEL FOR ADULT SEPTIC SHOCK, filed on Nov. 2, 2012; International Patent Application No. PCT/US13/25223, A MULTI-BIOMARKER-BASED OUTCOME RISK STRATIFICATION MODEL FOR PEDIATRIC SEPTIC SHOCK, filed on Feb. 7, 2013; International Patent Application No. PCT/US13/25221, A MULTI-BIOMARKER-BASED OUTCOME RISK STRATIFICATION MODEL FOR ADULT SEPTIC SHOCK, filed on Feb. 7, 2013; U.S. Provisional Application No. 61/908,613, TEMPORAL PEDIATRIC SEPSIS BIOMARKER RISK MODEL, filed on Nov. 25, 2013; International Patent Application No. PCT/US14/067438, TEMPORAL PEDIATRIC SEPSIS BIOMARKER RISK MODEL, filed on Nov. 25, 2014; U.S. patent application Ser. No. 15/998,427, SEPTIC SHOCK ENDOTYPING STRATEGY AND MORTALITY RISK FOR CLINICAL APPLICATION, filed on Aug. 15, 2018; U.S. Provisional Application No. 62/616,646, TEMPORAL ENDOTYPE TRANSITIONS REFLECT CHANGING RISK AND TREATMENT RESPONSE IN PEDIATRIC SEPTIC SHOCK, filed on Jan. 12, 2018; International Application No. PCT/US2017/032538, SIMPLIFICATION OF A SEPTIC SHOCK ENDOTYPING STRATEGY FOR CLINICAL APPLICATIONS, filed on May 12, 2017; U.S. Provisional Application No. 62/335,803, SIMPLIFICATION OF A SEPTIC SHOCK ENDOTYPING STRATEGY FOR CLINICAL APPLICATIONS, filed on May 13, 2016; U.S. Provisional Application No. 62/427,778, SIMPLIFICATION OF A SEPTIC SHOCK ENDOTYPING STRATEGY FOR CLINICAL APPLICATIONS, filed on Nov. 29, 2016; U.S. Provisional Application No. 62/428,451, SIMPLIFICATION OF A SEPTIC SHOCK ENDOTYPING STRATEGY FOR CLINICAL APPLICATIONS, filed on Nov. 30, 2016; U.S. Provisional Application No. 62/446,216, SIMPLIFICATION OF A SEPTIC SHOCK ENDOTYPING STRATEGY FOR CLINICAL APPLICATIONS, filed on Jan. 13, 2017; U.S. patent application Ser. No. 16/539,128, SEPTIC SHOCK ENDOTYPING STRATEGY AND MORTALITY RISK FOR CLINICAL APPLICATION, filed on Aug. 13, 2019; U.S. Provisional Application No. 62/764,831, ENDOTYPE TRANSITIONS DURING THE ACUTE PHASE OF PEDIATRIC SEPTIC SHOCK REFLECT CHANGING RISK AND TREATMENT RESPONSE, filed on Aug. 15, 2018; U.S. Provisional Application No. 63/149,744, A CONTINUOUS METRIC TO ASSESS THE INTERACTION BETWEEN ENDOTYPE ASSIGNMENT AND CORTICOSTEROID RESPONSIVENESS IN SEPTIC SHOCK, filed on Feb. 16, 2021; and International Patent Application No. PCT /US2022/016642, A CONTINUOUS METRIC TO ASSESS THE INTERACTION BETWEEN ENDOTYPE ASSIGNMENT AND CORTICOSTEROID RESPONSIVENESS IN SEPTIC SHOCK, filed on Feb. 16, 2022.

Unless otherwise noted, terms are to be understood according to conventional usage by those of ordinary skill in the relevant art.

As used herein, the term “sample” encompasses a sample obtained from a subject or patient. The sample can be of any biological tissue or fluid. Such samples include, but are not limited to, sputum, saliva, buccal sample, oral sample, blood, serum, mucus, plasma, urine, blood cells (e.g., white cells), circulating cells (e.g. stem cells or endothelial cells in the blood), tissue, core or fine needle biopsy samples, cell-containing body fluids, free floating nucleic acids, urine, stool, peritoneal fluid, and pleural fluid, tear fluid, or cells therefrom. Samples can also include sections of tissues such as frozen or fixed sections taken for histological purposes or micro-dissected cells or extracellular parts thereof. A sample to be analyzed can be tissue material from a tissue biopsy obtained by aspiration or punch, excision or by any other surgical method leading to biopsy or resected cellular material. Such a sample can comprise cells obtained from a subject or patient. In some embodiments, the sample is a body fluid that include, for example, blood fluids, serum, mucus, plasma, lymph, ascitic fluids, gynecological fluids, or urine but not limited to these fluids. In some embodiments, the sample can be a non-invasive sample, such as, for example, a saline swish, a buccal scrape, a buccal swab, and the like.

As used herein, “blood” can include, for example, plasma, serum, whole blood, blood lysates, and the like.

As used herein, the term “assessing” includes any form of measurement, and includes determining if an element is present or not. The terms “determining,” “measuring,” “evaluating,” “assessing” and “assaying” can be used interchangeably and can include quantitative and/or qualitative determinations.

As used herein, the term “monitoring” with reference to sepsis and/or necrotizing enterocolitis refers to a method or process of determining the severity or degree of sepsis and/or necrotizing enterocolitis or stratifying septic shock based on risk and/or probability of mortality. In some embodiments, monitoring relates to a method or process of determining the therapeutic efficacy of a treatment being administered to a patient.

As used herein, “outcome” can refer to an outcome studied. In some embodiments, “outcome” can refer to organ dysfunction and/or death after sepsis and/or necrotizing enterocolitis. In some embodiments, “outcome” can refer to two or more organ dysfunctions or death by day 7 of sepsis and/or necrotizing enterocolitis. In some embodiments, “outcome” can refer to day 7 cardiovascular, respiratory, renal, hepatic, hematologic, and neurologic dysfunction.

In some embodiments, “outcome” can refer to in hospital survival/mortality. In some embodiments, “outcome” can refer to 28-day survival/mortality. The importance of survival/mortality in the context of neonatal sepsis/necrotizing enterocolitis is readily evident. The common choice of 7-day mortality is a useful primary endpoint for interventional clinical trials involving critically ill patients. In some embodiments, an increased risk for a poor outcome indicates that a therapy has had a poor efficacy, and a reduced risk for a poor outcome indicates that a therapy has had a good efficacy. In some embodiments, “outcome” can refer to dysfunction of one or more, or two or more, organs after 1 day, 2 days, 3 days, 4 days, 5 days, 6 days, 7 days, 14 days, or 28 days. In some embodiments, “outcome” can include cardiovascular, respiratory, renal, hepatic, hematologic, and/or neurologic dysfunction. In some embodiments, “outcome” can refer to dysfunction in one or more organs including the heart, lungs, kidneys, liver, blood, and brain, and the like. In some embodiments, “outcome” can refer to resolution of organ failure after 7 days, 14 days, or 28 days, or limb loss. Although mortality/survival is obviously an important outcome, survivors have clinically relevant short- and long-term morbidities that impact quality of life, which are not captured by the dichotomy of “alive” or “dead.” In the absence of a formal, validated quality of life measurement tool for survivors of neonatal sepsis/necrotizing enterocolitis, resolution of organ failure can be used as a secondary outcome measure. For example, the presence or absence of new organ failure over one or more timeframes can be tracked. Organ failure is described herein in Example 1 (Methods). Specifically, cardiovascular, respiratory, renal, hepatic, hematologic, and neurologic failure can be tracked. As used herein, “outcome” can also refer to complicated course. Complicated course as defined herein relates to persistence of two or more organ failures at day one, day two, day three, day four, day five, day six, or day seven, of sepsis and/or necrotizing enterocolitis. Complicated course as defined herein can also relate to in hospital mortality and/or 28-day mortality.

As used herein, the terms “predicting outcome” and “outcome risk stratification” with reference to neonatal sepsis/necrotizing enterocolitis refers to a method or process of prognosticating a patient's risk of a certain outcome. In some embodiments, predicting an outcome relates to monitoring the therapeutic efficacy of a treatment being administered to a patient. In some embodiments, predicting an outcome relates to determining a relative risk of an adverse outcome (e.g. complicated course) and/or mortality. In some embodiments, the predicted outcome is associated with administration of a particular treatment or treatment regimen. Such adverse outcome risk and/or mortality can be high risk, moderate risk, moderate-high risk, moderate-low risk, or low risk. Alternatively, such adverse outcome risk can be described simply as high risk or low risk, corresponding to high risk of adverse outcome (e.g. complicated course) and/or mortality probability, or high likelihood of therapeutic effectiveness, respectively. In some embodiments of the present invention, adverse outcome risk can be determined via the biomarker-based mortality and/or complicated course risk stratification as described herein. In some embodiments, predicting an outcome relates to determining a relative risk of mortality and/or complicated course. Such mortality risk can be high risk, moderate risk, moderate-high risk, moderate-low risk, or low risk. Alternatively, such mortality risk can be described simply as high risk or low risk, corresponding to high risk of death or high likelihood of survival, respectively. As related to the terminal nodes of the decision trees described herein, a “high risk terminal node” corresponds to an increased probability of adverse outcome (e.g. complicated course) and/or mortality according to a particular treatment or treatment regimen, whereas a “low risk terminal node” corresponds to a decreased probability of adverse outcome (e.g. complicated course) and/or mortality according to a particular treatment or treatment regimen.

As used herein, the term “high risk clinical trial” refers to one in which the test agent has “more than minimal risk” (as defined by the terminology used by institutional review boards, or IRBs). In some embodiments, a high risk clinical trial is a drug trial.

As used herein, the term “low risk clinical trial” refers to one in which the test agent has “minimal risk” (as defined by the terminology used by IRBs). In some embodiments, a low risk clinical trial is one that is not a drug trial. In some embodiments, a low risk clinical trial is one that that involves the use of a monitor or clinical practice process. In some embodiments, a low risk clinical trial is an observational clinical trial.

As used herein, the terms “modulated” or “modulation,” or “regulated” or “regulation” and “differentially regulated” can refer to both up regulation (i.e., activation or stimulation, e.g., by agonizing or potentiating) and down regulation (i.e., inhibition or suppression, e.g., by antagonizing, decreasing or inhibiting), unless otherwise specified or clear from the context of a specific usage.

As used herein, the term “subject” refers to any member of the animal kingdom. In some embodiments, a subject is a human patient. In some embodiments, a subject is a pediatric patient. In some embodiments, a subject is a neonate pediatric patient. In some embodiments, a subject is hours, days, up to 28 days, up to one month old, up to 5 weeks old, tw months old, up to 6 months old, or up to 12 months old. In some embodiments, a subject is up to 12 months old, as long as the patient is still admitted to the newborn intensive care unit. In some embodiments, a pediatric patient is a patient under 18 years of age, while an adult patient is 18 or older.

As used herein, the terms “treatment,” “treating,” “treat,” and the like, refer to obtaining a desired pharmacologic and/or physiologic effect. The effect can be prophylactic in terms of completely or partially preventing a disease or symptom thereof and/or can be therapeutic in terms of a partial or complete cure for a disease and/or adverse effect attributable to the disease. “Treatment,” as used herein, covers any treatment of a disease in a subject, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease and/or relieving one or more disease symptoms. “Treatment” can also encompass delivery of an agent or administration of a therapy in order to provide for a pharmacologic effect, even in the absence of a disease or condition.

As used herein, the term “marker” or “biomarker” refers to a biological molecule, such as, for example, a nucleic acid, peptide, protein, hormone, and the like, whose presence or concentration can be detected and correlated with a known condition, such as a disease state. It can also be used to refer to a differentially expressed gene whose expression pattern can be utilized as part of a predictive, prognostic or diagnostic process in healthy conditions or a disease state, or which, alternatively, can be used in methods for identifying a useful treatment or prevention therapy.

As used herein, the term “expression levels” refers, for example, to a determined level of biomarker expression. The term “pattern of expression levels” refers to a determined level of biomarker expression compared either to a reference (e.g. a housekeeping gene or inversely regulated genes, or other reference biomarker) or to a computed average expression value (e.g. in DNA-chip analyses). A pattern is not limited to the comparison of two biomarkers but is more related to multiple comparisons of biomarkers to reference biomarkers or samples. A certain “pattern of expression levels” can also result and be determined by comparison and measurement of several biomarkers as disclosed herein and display the relative abundance of these transcripts to each other.

As used herein, a “reference pattern of expression levels” refers to any pattern of expression levels that can be used for the comparison to another pattern of expression levels. In some embodiments of the invention, a reference pattern of expression levels is, for example, an average pattern of expression levels observed in a group of healthy or diseased individuals, serving as a reference group.

As used herein, the term “decision tree” refers to a standard machine learning technique for multivariate data analysis and classification. Decision trees can be used to derive easily interpretable and intuitive rules for decision support systems.

In particular aspects, the Pediatric Sepsis Biomarker Risk Model (PERSEVERE) [9] can be used to estimate baseline risk of mortality among neonates with sepsis and/or necrotizing enterocolitis. According to particular aspects of the present invention, validated neonatal-specific prognostic tools called nPERSEVERE-1 and nPERSEVERE-2 have been developed, using decision tree methodology to predict mortality at discharge in neonates who experienced sepsis, using the PERSEVERE biomarkers. In some embodiments directed to exclusively neonatal population aspects (nPERSEVERE-1 and/or nPERSEVERE-2), a neonatal-specific decision tree is provided using two or more PERSEVERE biomarkers. In some embodiments directed to exclusively neonatal population aspects (nPERSEVERE-1), a neonatal-specific decision tree is provided using the PERSEVERE biomarkers IL-8 and CCL3. In some embodiments directed to exclusively neonatal population aspects (nPERSEVERE-2), a neonatal-specific decision tree is provided using the PERSEVERE biomarkers IL-8 and MMP8, as well as platelet count. In some embodiments directed to exclusively neonatal population aspects (nPERSEVERE-2), a determination that a patient is low risk can be made based on a single biomarker, namely IL-8.

Various methods as used herein have been previously described (see, for example, Wong H R, Cvijanovich N Z, Anas N, Allen G L, Thomas N J, Bigham M T, Weiss S L, Fitzgerald J, Checchia P A, Meyer K et al.: Developing a clinically feasible personalized medicine approach to pediatric septic shock. Am J Respir Crit Care Med 2015, 191(3):309-315; Wong 2016; Wong 2012). For example, the 100 endotype-defining genes and the four housekeeping genes were previously reported (Wong 2015). The four housekeeping genes were used to normalize the NanoString-derived expression data: β-2-microglobulin (B2M), folylpolyglutamate synthase (FPGS), 2,4-dienoyl CoA reductase 1 (DECR1), and peptidylprolyl isomerase B (PPIB) (Wong 2015). Expression values were normalized to the geometric mean of the housekeeping genes. Gene expression was quantified using the NanoString nCounter platform (NanoString Technologies, Seattle, Wash.) (Wong 2015). The endotype assignment procedure was also previously detailed (Wong 2015).

As described herein, biomarkers can be used at the time of evaluation for neonatal sepsis (blood culture acquisition) to identify neonates with high baseline mortality risk. This is an important step towards precision medicine in neonatal sepsis.

Prospective cohorts of neonates were studied. Patients were admitted to Cincinnati Children's Hospital Medical Center NICU and the University of Cincinnati Medical Center NICU. Patients were enrolled if they met criteria for sepsis according to Wynn et al., 2010 or had NEC stage II or greater according to Bell et al., 1978. Neonates were excluded if they only received 48 hours of antibiotics or found to have a lethal diagnosis. Residual serum at the time of sepsis/NEC evaluation was obtained for biomarker analysis using Luminex platform. Biomarker analysis was done on serum samples obtained at the time of evaluation for the event The primary outcome was mortality at discharge and the secondary outcome was complicated course of illness (defined as death or the persistence of 2 or more organ dysfunction on day 7 of illness). Statistical analysis was done using Prism v9.0 and classification tree analysis was done using Salford Predictive Modeler v.8.0.

The first neonatal specific analysis (Cohort 1) included 86 events. Six (6) were excluded for discontinuation of antibiotics at 48 hours and two (2) were excluded for terminal diagnoses. The included neonates (71) had 64 sepsis events and 14 NEC events with an overall mortality rate of 18.3% (Table 1). If a neonate had multiple events, only the last event captured was included in the analysis.

PERSEVERE II classified neonates into a higher risk group (mortality 29.4%, complicated course 41.8%) and lower risk group (mortality 8.1%, complicated course 16.2%). It was 77% sensitive and 59% specific for mortality, p 0.03 (Table 2).

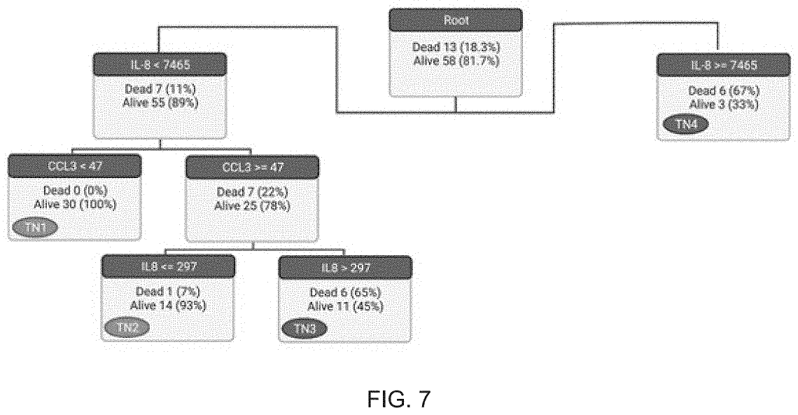

A new tree for neonates (nPERSEVERE-1) was derived using Classification and Regression Tree methodology (FIG. 7). The decision tree shown in FIG. 7 includes the cytokines IL8 and CCL3 and has 4 terminal nodes. The cytokine level was obtained from serum samples using Luminex technology, as referenced in the Methods section. Briefly, cytokines were measured using a multiplex magnetic bead platform designed by EMD Millipore Corporation (Millipore Sigma, MA, USA) specifically for PERSEVERE. Serum levels of biomarkers were obtained using a Luminex 100/200 plate reader (Luminex Corporation, Austin, Tex.), according to the manufacturer's protocol.

nPERSEVERE-1 had an AUROC of 0.89, with 92% sensitivity and 76% specificity for estimating the risk of mortality. Upon 10-fold cross validation the summary AUROC was 0.73. Among patients classified to terminal nodes 3 and 4, the mortality rate was 46% and the complicated course rate was 66%. In contrast, among patients classified to terminal nodes 1 and 2, the mortality rate was 2% and complicated course rate was 13%, p<0.0001.

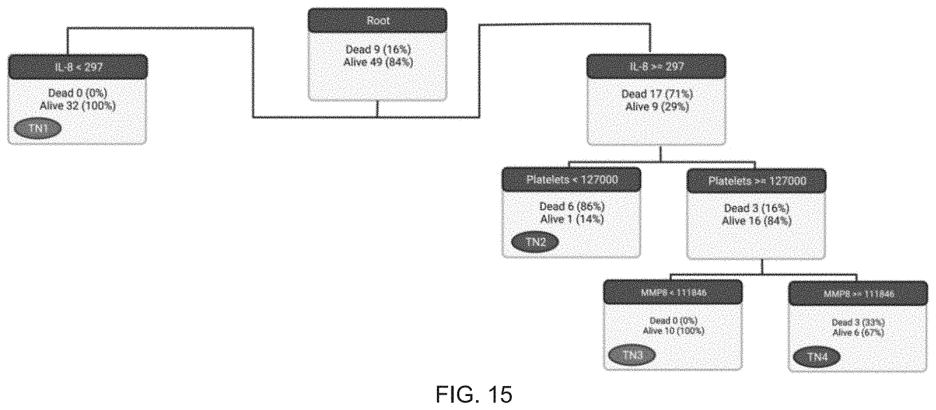

The second neonatal specific analysis (Cohort 2) included 59 neonates, with a mortality rate of 15.3%. PERSEVERE II was 67% sensitive and 59% specific for mortality, p 0.27. Amongst PERSEVERE II biomarkers, IL-8 showed good prognostic performance for mortality prediction with a cutoff of 300 pg/mL (sensitivity 100%, specificity 65%, negative predictive value 100%, AUC 0.87, p 0.0003). A new decision tree was derived that is neonate specific (nPERSEVERE-2), shown in FIG. 15. nPERSEVERE-2 has improved performance compared to IL-8 (sensitivity 100%, specificity 86%, negative predictive value 100%, AUC 0.95, p<0.0001).

Accordingly, IL-8, nPERSEVERE-1, and nPERSEVERE-2 demonstrated good prognostic performance in cohorts of neonates with sepsis. Moving towards precision medicine in sepsis, this work provides an important tool for clinical trial prognostic enrichment.

According to particular aspects of the present invention, nPERSEVERE-1, and nPERSEVERE-2 have utility for identifying neonates at higher risk of dying from sepsis and NEC who could benefit from high-risk therapies and aid in clinical trial enrichment.

### Determining Neonate Risk of Mortality and/or Complicated Course Due to Sepsis and/or Necrotizing Enterocolitis

Reliable risk stratification has numerous clinical applications. These include better-informed allocation of critical care resources, appropriate selection of patients for higher risk and more costly therapies, and for benchmarking outcomes. Additionally, risk stratification can serve as a prognostic enrichment tool to greatly enhance efficiency of clinical trials. Reliable risk stratification of patients with sepsis and/or necrotizing enterocolitis can be a challenging task due to significant patient heterogeneity.

PERSEVERE is a multi-biomarker decision tree that is now validated to reliably estimate baseline risk of mortality among children with septic shock. PERSEVERE was developed as a tool to identify pediatric patients with sepsis at high mortality risk in the PICU based on five serum biomarkers, CCL3 (CC Chemokine Ligand 3), IL-8 (Interleukin-8), HSPA1b (Heatshock Protein A1b), GZMB (Granzyme B), and MMP-8 (Matrix Metallopeptidase 8), and platelet count [9]. These biomarkers were chosen after identifying gene probes that were differentially expressed in pediatric patients with septic shock that did not survive. The specific measurable serum proteins have also been reported to have a role in the pathophysiology of septic shock [10]. This promising prognostic tool has been validated in pediatric and adult cohorts with good performance in identifying high-risk patients early in the disease course [9, 11-13].

The most recent iteration of PERSEVERE is called PERSEVERE II [9], which is a validated pediatric prognostic tool and includes platelet count in addition to the abovementioned biomarkers, and it has shown good performance in a large pediatric cohort of 461 patients. PERSEVERE II is 86% sensitive and 69% specific for mortality with a negative predictive value of 97% and an area under the curve of 0.83.

The majority of conducted biomarker research in neonatal sepsis is focused on early diagnosis of culture positive sepsis, but this has yet to change clinical practice. As described herein, the utility of PERSEVERE II was assessed, which uses decision tree methodology to predict mortality at discharge in neonates who experienced sepsis or necrotizing enterocolitis. The finding that biomarkers can be used early in the course of neonatal sepsis to identify neonates with high baseline mortality risk is an important step towards precision medicine in neonatal sepsis.

Given the paucity of prognostic biomarker research in neonatal sepsis, a prospective study was conducted in a dual-center cohort of neonates with sepsis or necrotizing enterocolitis admitted between June 2020 and December 2021 to assess the utility of PERSEVERE II and its biomarkers as possible prognostic tools in neonatal sepsis. Biomarker analysis was done on whole blood samples obtained at the time of evaluation for the event. Since neonates with necrotizing enterocolitis have a clinical phenotype that resembles neonates with sepsis and septic shock, neonates with stage II or greater necrotizing enterocolitis were included in this analysis as well. Because for all ongoing research in neonatal sepsis, there is no consensus definition for neonatal sepsis comparable to the ones that exist for adult and pediatric patients [23, 24], the present study overcomes this by using a definition that was proposed by an expert in the field allowing for prospective enrollment at the time of evaluation without relying on blood culture results [15], which usually occurs hours to days after the initial suspicion.

In Cohort 1, with 71 neonates with a mortality rate of 18.3%, PERSEVERE II was 77% sensitive and 59% specific for mortality, p 0.03. Amongst PERSEVERE II biomarkers, IL-8 showed good prognostic performance for mortality prediction with a cutoff of 300 pg/mL (sensitivity 92%, specificity 62%, negative predictive value 97%, AUC 0.83, p 0.004). A new decision tree that is neonate specific was derived (termed nPERSEVERE-1) with improved performance compared to IL-8 (sensitivity 92%, specificity 76%, negative predictive value 98%, AUC 0.89, p<0.0001).

In Cohort 2, with 59 neonates with a mortality rate of 15.3%, PERSEVERE II was 67% sensitive and 59% specific for mortality, p 0.27. Amongst PERSEVERE II biomarkers, IL-8 showed good prognostic performance for mortality prediction with a cutoff of 300 pg/mL (sensitivity 100%, specificity 65%, negative predictive value 100%, AUC 0.87, p0.0003). A new decision tree that is neonate specific was derived (termed nPERSEVERE-2) with improved performance compared to IL-8 (sensitivity 100%, specificity 86%, negative predictive value 100%, AUC 0.95, p<0.0001).

Thus, IL-8, nPERSEVERE-1, and nPERSEVERE-2 demonstrated good prognostic performance in a small cohort of neonates with sepsis and necrotizing enterocolitis. Moving towards precision medicine, this study provides an important tool for clinical trial prognostic enrichment.

### Additional Patient Information

The demographic data, clinical characteristics, and/or results from other tests or indicia of neonatal sepsis/necrotizing enterocolitis can affect the patient's outcome risk. Accordingly, such demographic data, clinical characteristics, and/or results from other tests or indicia of neonatal sepsis/necrotizing enterocolitis can be incorporated into the methods described herein which allow for stratification of an individual neonate in order to determine the patient's outcome risk. Such demographic data, clinical characteristics, and/or results from other tests or indicia of neonatal sepsis/necrotizing enterocolitis can also be used in combination with the methods described herein which allow for stratification of individual pediatric patients in order to determine the patient's outcome risk.

Such neonatal patient demographic data can include, for example, the patient's gestational age at birth, chronological age, race, gender, and the like. In some embodiments, the nPERSEVERE-1 and/or nPERSEVERE-2 biomarker-based mortality and/or complicated course risk stratification described herein can incorporate or be used in combination with the patient's age, gestational age at birth, birth weight, race, and/or gender to determine an outcome risk.

Such patient clinical characteristics and/or results from other tests or indicia of neonatal sepsis/necrotizing enterocolitis can include, for example, the patient's co-morbidities and/or neonatal sepsis/necrotizing enterocolitis causative organism, and the like.

Patient co-morbidities observed for neonates can include, for example, developmental delay, DiGeorge syndrome, Down syndrome, drowning, end stage renal disease, glycogen storage disease type 1, hydrocephalus, liver failure, metaleukodystrophy, mitochondrial disorder, multiple congenital anomalies, Pallister Killian syndrome, Prader-Willi syndrome, requirement for chronic dialysis, sarcoma, severe combined immune deficiency, short gut syndrome, sickle cell disease, sleep apnea, subglottic stenosis, tracheal stenosis, traumatic brain injury, trisomy 18, VATER Syndrome, and the like. Any one or more of the above patient co-morbidities can be indicative of the presence or absence of chronic disease in the patient.

Neonatal sepsis/necrotizing enterocolitis causative organisms can include, for example, Acinetobacter baumannii, Adenovirus, Bacteroides species, Candida species, Capnotyophaga jenuni, Cytomegalovirus, Enterobacter cloacae, Enterococcus faecalis, Escherichia coli, Herpes simplex virus, Human metapneumovirus, Influenza A, Klebsiella pneumonia, Micrococcus species, mixed bacterial infection, Moraxella catarrhalis, Neisseria meningitides, Parainfluenza, Pseudomonas species, Serratia marcescens, Staphylococcus aureus, Streptococcus agalactiae, Streptococcus milleri, Streptococcus pneumonia, Streptococcus pyogenes, unspecified gram negative rods, unspecified gram positive cocci, and the like.

In some embodiments, the biomarker-based mortality and/or complicated course risk stratification as described herein can incorporate the patient's co-morbidities to determine an outcome risk and/or mortality probability. In some embodiments, the biomarker-based mortality and/or complicated course risk stratification as described herein can incorporate the patient's neonatal sepsis/necrotizing enterocolitis causative organism to determine an outcome risk and/or mortality probability.

In some embodiments, the biomarker-based mortality and/or complicated course risk stratification as described herein can be used in combination with the patient's co-morbidities to determine an outcome risk and/or mortality probability. In some embodiments, the biomarker-based mortality and/or complicated course risk stratification as described herein can be used in combination with the patient's sepsis and/or necrotizing enterocolitis causative organism to determine an outcome risk and/or mortality probability.

### PERSEVERE, PERSEVERE II, and Other Population-Based Risk Scores

As mentioned previously, the PERSEVERE and PERSEVERE II models for estimating baseline mortality risk in children with septic shock were previously derived and validated. PERSEVERE and PERSEVERE II based on a panel of 12 serum protein biomarkers measured from blood samples obtained during the first 24 hours of a septic shock diagnosis, selected from among 80 genes having an association with mortality risk in pediatric septic shock. Of those 12 serum biomarkers, the derived and validated PERSEVERE and PERSEVERE II models are based on Interleukin-8 (IL-8), Heat shock protein 70 kDA (HSP70), C-C Chemokine ligand 3 (CCL3), C-C Chemokine ligand 4 (CCL4), Granzyme B (GZMB), Interleukin-1 α(IL-1α), and Matrix metallopeptidase 8 (MMP8). PERSEVERE additionally takes patient age into account.

In some embodiments of the present invention, a patient sample is analyzed for one or more of the PERSEVERE serum protein biomarkers IL-8, MMP8, and CCL3. In some embodiments of the present invention, a patient sample is analyzed to determine platelet count.

In some embodiments of the present invention, the PERSEVERE or PERSEVERE II mortality probability stratification can be used in combination with the nPERSEVERE-1 and/or nPERSEVERE-2 biomarker-based mortality and/or complicated course risk stratification as described herein. In some embodiments, the nPERSEVERE-1 and/or nPERSEVERE-2 biomarker-based mortality and/or complicated course risk stratification, as described herein, can be used in combination with a patient endotyping strategy and/or Z score determination. In some embodiments, the combination of the nPERSEVERE-1 and/or nPERSEVERE-2 biomarker-based mortality and/or complicated course risk stratification, with an endotyping strategy and/or Z score determination, can be used to determine an appropriate treatment regimen for a patient. For example, such combinations can be used to identify which patients are more likely to benefit from corticosteroids.

A number of additional models that generate mortality prediction scores based on physiological variables have been developed to date. These can include the PRISM, Pediatric Index of Mortality (PIM), and/pediatric logistic organ dysfunction (PELOD) models, and the like.

Such models can be very effective for estimating population-based outcome risks but are not intended for stratification of individual patients. The methods described herein which allow for stratification of individual patients can be used alone or in combination with one or more existing population-based risk scores.

In some embodiments, the nPERSEVERE-1 and/or nPERSEVERE-2 biomarker-based mortality and/or complicated course risk stratification described herein can be used with one or more additional population-based risk scores. In some embodiments, the nPERSEVERE-1 and/or nPERSEVERE-2 biomarker-based mortality and/or complicated course risk stratification described herein can be used in combination with patient demographic data, such as gestational age, birth weight, and the like.

### High Risk Therapies

High risk, invasive therapeutic and support modalities can be used to treat sepsis and/or necrotizing enterocolitis. The methods described herein which allow for the patient's outcome risk to be determined can help inform clinical decisions regarding the application of high risk therapies to specific pediatric patients, based on the patient's outcome risk.

High risk therapies include, for example, extracorporeal membrane oxygenation/life support, plasmapheresis, pulmonary artery catheterization, high volume continuous hemofiltration, and the like. High risk therapies can also include non-corticosteroid therapies, e.g. alternative therapies and/or high risk therapies. In particular, patients at high risk of mortality and/or complicated course can be treated with immune enhancing and/or modulating therapies, such as, for example, GMCSF, IVIG, anit-IL-8, anti-IL-2, interleukin-7, anti-PD-1, and the like.

In some embodiments, individualized treatment can be provided to a neonate by selecting a neonate classified as high risk by the methods described herein for one or more high risk therapies. In some embodiments, individualized treatment can be provided to a neonate by excluding a neonate classified as low risk from one or more high risk therapies.

Certain embodiments of the invention include using quantification data from a gene-expression analysis and/or from a protein, mRNA, and/or DNA analysis, from a sample of blood, urine, saliva, broncho-alveolar lavage fluid, or the like. Embodiments of the invention include not only methods of conducting and interpreting such tests but also include reagents, compositions, kits, tests, arrays, apparatuses, processing devices, assays, and the like, for conducting the tests. The compositions and kits of the present invention can include one or more components which enable detection of the biomarkers disclosed herein and combinations thereof and can include, but are not limited to, primers, probes, cDNA, enzymes, covalently attached reporter molecules, and the like.

Diagnostic-testing procedure performance is commonly described by evaluating control groups to obtain four critical test characteristics, namely positive predictive value (PPV), negative predictive value (NPV), sensitivity, and specificity, which provide information regarding the effectiveness of the test. The PPV of a particular diagnostic test represents the proportion of positive tests in subjects with the condition of interest (i.e. proportion of true positives); for tests with a high PPV, a positive test indicates the presence of the condition in question. The NPV of a particular diagnostic test represents the proportion of negative tests in subjects without the condition of interest (i.e. proportion of true negatives); for tests with a high NPV, a negative test indicates the absence of the condition. Sensitivity represents the proportion of subjects with the condition of interest who will have a positive test; for tests with high sensitivity, a positive test indicates the presence of the condition in question. Specificity represents the proportion of subjects without the condition of interest who will have a negative test; for tests with high specificity, a negative test indicates the absence of the condition.

The threshold for the disease state can alternatively be defined as a 1-D quantitative score, or diagnostic cutoff, based upon receiver operating characteristic (ROC) analysis. The quantitative score based upon ROC analysis can be used to determine the specificity and/or the sensitivity of a given diagnosis based upon subjecting a patient to a decision tree described herein in order to predict an outcome for a neonate with neonatal sepsis/necrotizing enterocolitis.

The correlations disclosed herein, between pediatric patient sepsis biomarker levels and/or mRNA levels and/or gene expression levels and/or protein expression levels, provide a basis for conducting a diagnosis of sepsis and/or necrotizing colitis, or for conducting a stratification of patients with sepsis and/or necrotizing colitis, or for enhancing the reliability of a diagnosis of sepsis and/or necrotizing colitis by combining the results of a quantification of a sepsis biomarker with results from other tests or indicia of neonatal sepsis/necrotizing enterocolitis, or for determining an appropriate treatment regimen for a neonate with sepsis and/or necrotizing colitis. For example, the results of a quantification of one biomarker could be combined with the results of a quantification of one or more additional biomarker, protein, cytokine, mRNA, or the like. Thus, even in situations in which a given biomarker correlates only moderately or weakly with neonatal sepsis/necrotizing enterocolitis, providing only a relatively small PPV, NPV, specificity, and/or sensitivity, the correlation can be one indicium, combinable with one or more others that, in combination, provide an enhanced clarity and certainty of diagnosis. Accordingly, the methods and materials of the invention are expressly contemplated to be used both alone and in combination with other tests and indicia, whether quantitative or qualitative in nature.

Having described the invention in detail, it will be apparent that modifications, variations, and equivalent embodiments are possible without departing from the scope of the invention defined in the appended claims. Furthermore, it should be appreciated that all examples in the present disclosure are provided as non-limiting examples.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### Methods for Examples 2-13

The methods used in Examples 2-13 are summarized below:

### Study Population.

This prospective cohort study was approved by the institutional review board at Cincinnati Children's Hospital Medical Center and the University of Cincinnati prior to data and specimen collection. The study was approved with waiver of consent given that whole blood was obtained from residual samples in the clinical laboratory and the study protocol did not alter or inform clinical care. Neonates were enrolled from the Cincinnati Children's Hospital Medical Center level IV NICU and the level III NICU at the University of Cincinnati Medical Center between June 2020 to December 2021. All neonates who had a complete blood count performed were screened for enrollment using Vigilanz reporting system (Vigilanz Corp, MN, USA). Clinical and laboratory data was obtained and stored in a secured RedCap (Research Electronic Data Capture; Vanderbilt University; Nashville, Tenn.) database.

### Cohort Enrollment.

Members of the study identified qualifying infants using Vigilanz (Vigilanz Corp, St. Louis Park, Minn.) who had a whole blood sample collected within the last 24 hours. Neonates who qualified for the study were enrolled, and the clinical core laboratory at the admitting hospital was contacted to obtain the residual sample that was collected from the patient at the time of evaluation for sepsis. Whole blood samples were centrifuged upon collection and serum was frozen and stored in the Critical Care Division Laboratories until analysis was performed.

Two separate cohorts were studied, termed “Cohort 1” and “Cohort 2” herein.

Neonates were enrolled if they fit the following criteria:

Suspected to have an infection as evident by obtaining whole blood counts, initiating antibiotic therapy, and with evidence of systemic inflammatory response by having two of the following (one must be number 1 or 2):

1) Leukocytosis, or leukopenia, or immature shift, or elevated CRP in preterm infants only;

2) Temperature instability;

3) Elevated respiratory rate for greater than 2 hours;

4) Elevated or depressed heart rate for greater than 2 hours.

Cohort 1 additionally enrolled neonates who were confirmed to have necrotizing enterocolitis stage II or greater based on radiographic evidence of pneumatosis intestinalis or portal venous gas or free air on at least two abdominal films as per the radiologist interpretation and being NPO with antibiotic for at least 7 days. Cohort 2 did not enroll such neonates.

Inclusion Criteria:

1) Neonates who are admitted to the intensive care unit who had a whole blood sample obtained for a suspected infection.

Exclusion Criteria:

1) Infants with congenital cardiac defects requiring early surgical intervention (within the first week of life);

2) Infants with a lethal chromosomal anomaly;

3) Infants who underwent surgical intervention within 7 days;

4) If the residual samples are older than 72 hours.

The neonate was assumed to not have an organ-specific dysfunction if the clinical team did not obtain the laboratory test that is used to define that dysfunction. Neonates were excluded if antibiotics were discontinued before 48 hours or if the neonate was found to have an underlying lethal diagnosis or a cardiac defect requiring intervention in the neonatal period. Infants with congenital anomalies were not excluded.

The primary outcome of the study was in hospital mortality. Secondary outcomes were the occurrence of complicated course (defined as in hospital mortality or 2 or more organ dysfunctions on day 7 of illness), vasopressor use, and duration of vasopressor use.

### Clinical Characteristics.

Patient clinical conditions were established according to the parameters listed below.

Sepsis. Suspected infection plus SIRS (Systemic Inflammatory Response Syndrome).

Term infants: Presence of at least two (one must be abnormal leukocyte count or abnormal temp) of the following:

a) Core Temp>38 C or <36 C;

b) Tachycardia (Greater than 180 bpm for greater than 2 hours) or bradycardia (less than 80 for greater than 2 hours);

c) Elevated respiratory rate or mechanical ventilation not related to anesthesia;

d) Leukocyte count>20,000 or <5,000 OR immature neutrophils>10%. Syndrome).

Preterm infants: Presence of at least two (one must be abnormal leukocyte count or abnormal temp) of the following:

a) Core Temp>38 C or <36 C;

b) Tachycardia (Greater than 180 bpm for greater than 2 hours) or bradycardia (less than 80 for greater than 2 hours);

c) Elevated respiratory rate (greater than 90 per minute for greater than 2 hours) or mechanical ventilation not related to anesthesia;

d) Leukocyte count>20,000 or <5,000 OR immature neutrophils>20% OR CRP>1 mg/dL.

Septic shock. Sepsis AND cardiovascular organ dysfunction .

Severe sepsis. Sepsis AND one of the following: cardiovascular dysfunction OR two or more other organ dysfunction.

Cardiac, respiratory, hepatic, neurologic, and hematologic dysfunctions were defined as suggested by Wynn et al., 2010 [15]. Renal dysfunction was defined as acute kidney injury according to KIDGO modified neonatal definitions [16]. Organ injury data was collected over the course of 7 days from the time of enrollment. The neonate was assumed to not have an organ-specific dysfunction if the clinical team did not obtain the laboratory test that is used to define that dysfunction.

Cardiac Dysfunction.

Term Infants: despite isotonic fluids>40 cc/kg in one hour, having hypotension as having two consecutive MAP readings according to the table below OR need of vasoactive drug OR two of (unexplained metabolic acidosis, increased lactic acid>2 times upper normal, urine output less than 0.5 mL/kg/hr, capillary refill>5 seconds).

Preterm infants: despite isotonic fluids>40 cc/kg in one hour (>10 cc/kg for infants less than 32 weeks), having hypotension OR need of vasoactive drug OR MAP<30 mmHg and delayed capillary refill>3 seconds, OR two of (unexplained metabolic acidosis, increased lactic acid>2 times upper normal, urine output less than 0.5 mL/kg/hr, capillary refill>4 seconds)

Respiratory Dysfunction.

Term infants: PaCO2>65 torr or 20 torr over baseline PaCO2 OR need for FiO2>50% to maintain saturations above 92% OR need for non-elective intubation.

Preterm infants: PaCO2>65 torr or 20 mmHg over baseline PaCO2 OR need for FiO2 >50% to maintain saturations above 92% (88% for infants<32 weeks) OR need for non-elective intubation.

Neurologic Dysfunction.

Term and preterm infants: Acute change in mental status.

Hematologic Dysfunction.

Term and preterm infants: Platelet count<80,000/mm or a decline greater than 50% from highest value in the last three days OR INR>2.

Renal dysfunction. Term and preterm infants: Absolute serum creatinine rise greater than 0.3 mg/dL OR rise in serum creatinine that is greater than 50% of lowest baseline OR urine output less than 1 mL/kg/hr for 24 hours.

Hepatic dysfunction. Term and preterm infants: ALT 2 times upper normal limit for age or 50% increase from baseline.

Complicated course. Persistence of 2 or more organ dysfunction at 7 days from the initial evaluation point.

### Serum Samples and Biomarker Assays.

Whole blood samples were obtained at the time of evaluation for sepsis by identifying the time when blood culture was obtained. Samples were collected from the clinical laboratory within 72 hours of acquisition, then centrifuged for 5 minutes at 500 G. Supernatant was isolated and stored at −80° C. Biomarkers were measured using a multiplex magnetic bead platform designed by EMD Millipore Corporation (Millipore Sigma, MA, USA) specifically for PERSEVERE. Concentrations of markers were obtained using a Luminex 100/200 plate reader (Luminex Corporation, Austin, Tex.) according to the manufacturer's protocol.

### Statistical Analysis.

Comparisons between survivors and non-survivors were performed using the Fisher's exact test or the Mann-Whitney U test when appropriate. The PERSEVERE II classification tree [9] was used to stratify patients into terminal nodes that were deemed high or low risk. High- and low-risk patient comparisons were done using the Fisher's exact test or the Mann-Whitney test when appropriate. Prism v9.0 was used for generating receiving-operator-curves and calculating the area under the curve. RStudio v1.4 (RStudio Team, MA, USA) and Salford Predictive Modeler v6.6 (Salford Systems, San Diego, Calif., USA) were used to derive and validate the new decision tree. The new tree was derived using RPART package in R studio by instructing the algorithm to classify neonates according to mortality outcome and to continue branching until 5% of the cohort is in the terminal node using all available PERSEVERE II serum biomarker levels and gestational age at birth.

### Example 2

### Cohort 1 Demographics

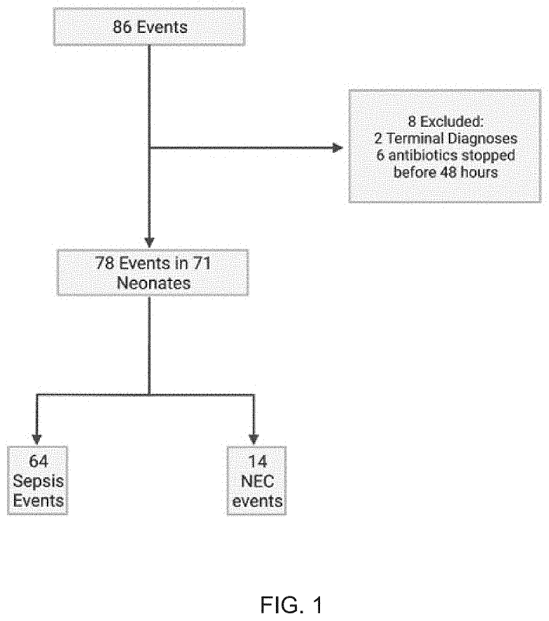

In total, Cohort 1 evaluated 86 events were evaluated for enrollment. 2 neonates were excluded for terminal diagnoses, and 6 neonates were excluded for discontinuation of antibiotics within 48 hours of evaluation. 71 neonates were included in the analysis who had 64 sepsis and 14 NEC events for a total of 78 events. For neonates who had multiple sepsis events during their NICU stay (n=4), only the last event they had was included in this analysis (FIG. 1). The overall mortality rate was 18.3%.

Overall, survivors and non-survivors were comparable in their characteristics. Although there was no statistical difference between both groups in all variables, including culture positivity rate in sepsis cases, there was a trend for smaller birth weight and earlier gestational age in non-survivors (Table 1).

TABLE 1
Cohort 1 Demographics. All characteristics were similar.

| | Mortality n = 13 | | Survival n = 58 | | |
|---|---|---|---|---|---|
| Characteristic | (18.3%) | | (81.7%) | | p Value |
| Female n (%) | 8 | (61.5%) | 27 | (46.6%) | 0.372^{a} |
| Male n (%) | 5 | (38.5%) | 31 | (53.4%) | |
| Black n (%) | 4 | (30.7%) | 18 | (31.0%) | >0.999 ^{a, b} |
| White n (%) | 7 | (53.8%) | 37 | (63.8%) | |
| Others n (%) | 2 | (15.5%) | 3 | (5.20%) | |
| Congenital | 3 | (23.1%) | 24 | (41.3%) | 0.344^{a} |
| Anomalies | | | | | |
| n (%) | | | | | |
| GA at Birth | 27 | (25-36) | 34 | (26-37) | 0.125^{c} |
| (IQR) weeks | | | | | |
| Birth Weight | 1030 | (557-1770) | 1664 | (820-2901) | 0.074^{c} |
| (range) gm | | | | | |
| Culture | 7/9 | (77.8%) | 42/48 | (87.5%) | 0.599^{a} |
| Positive in | | | | | |
| Sepsis n (%) | | | | | |

^{a}p value calculated using Fisher's exact test.
^{b}p value calculated comparing neonates who are black vs. white.
^{c}p value calculated using Mann-Whitney U test.

### Example 3

### Performance of PERSEVERE II for Neonates with Sepsis and Necrotizing Enterocolitis in Cohort 1

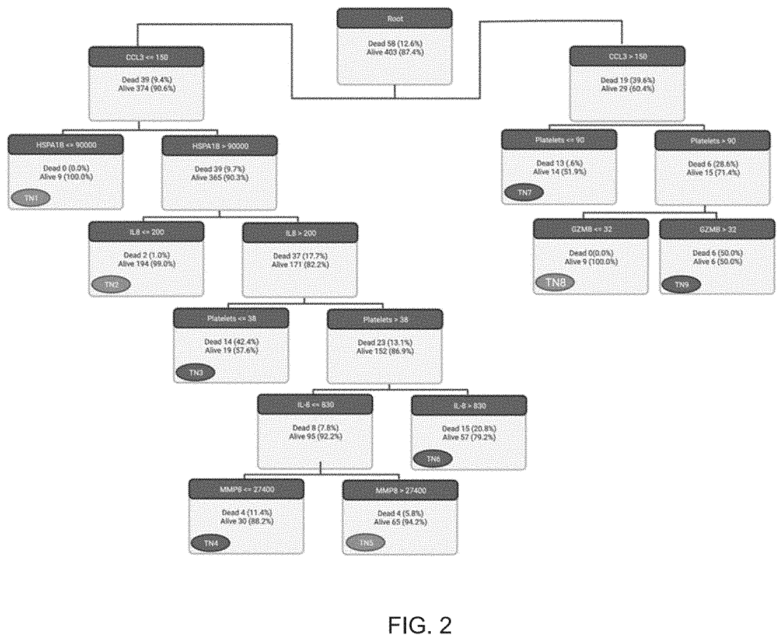

Neonates in Cohort 1 were classified according to the PERSEVERE II decision tree as described previously (FIG. 2), and neonates who were classified to terminal nodes one, two, and eight were considered low risk (predicted survivors), and those classified to terminal nodes three, four, six, seven, and nine were considered high risk (predicted non-survivors). The entire Cohort 1 had a mortality rate of 18.3%, and those who were classified as low risk had a mortality rate of 8.1% while high risk patients had a rate of 29.4%. PERSEVERE II was 77% sensitive and 59% specific for mortality, p 0.03 (Table 2). High risk patients also had higher complicated course rate compared to low-risk ones, 50% vs. 16.2% respectively, p 0.004.

TABLE 2
Performance of Candidate Predictors of Mortality in Neonatal Sepsis in Cohort
1. Summary of candidate predictors. IL-8 is superior to PERSEVERE II with improved
sensitivity, specificity, negative predictive value, and area under the curve.
nPERSEVERE-1 provides improved specificity and area under the curve.

| | PERSEVERE | IL-8 | nPERSEVERE-1 |
|---|---|---|---|
| Sensitivity (95% CI) | 77% (49.7% to 91.8%) | 92% (67% to 100%) | 92% (67% to 100%) |
| Specificity (95% CI) | 58.6% (45.8% to 70.4%) | 62% (49% to 73%) | 76% (64% to 85%) |
| Negative Predictive Value (95% CI) | 91.9% (78.7% to 97.2%) | 97% (86% to 100%) | 98% (87% to 100%) |
| Likelihood Ratio | 1.9 | 2.4 | 3.8 |
| AUC (95% CI) | 0.67 (0.53-0.82) | 0.83 (0.72 to 0.94) | 0.89 (0.81-0.97) |
| Fisher's Exact p Value | 0.03 | 0.0004 | <0.0001 |

### Example 4

### IL-8 is a Candidate Marker for Mortality Prediction in Cohort 1

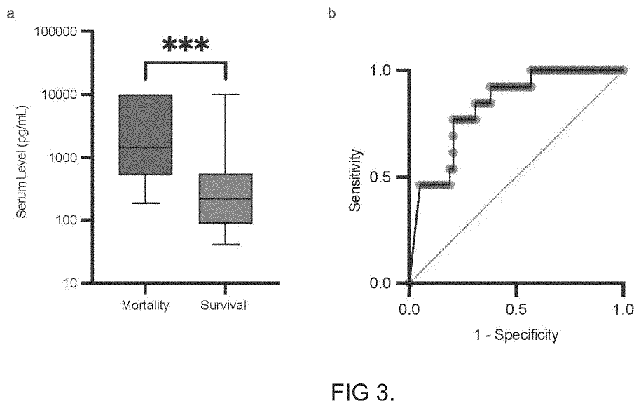

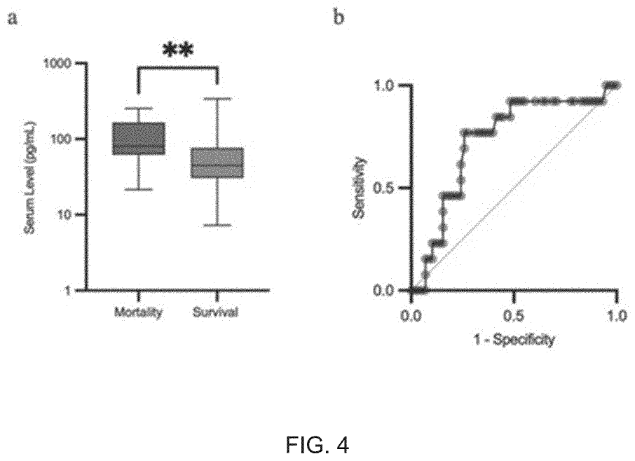

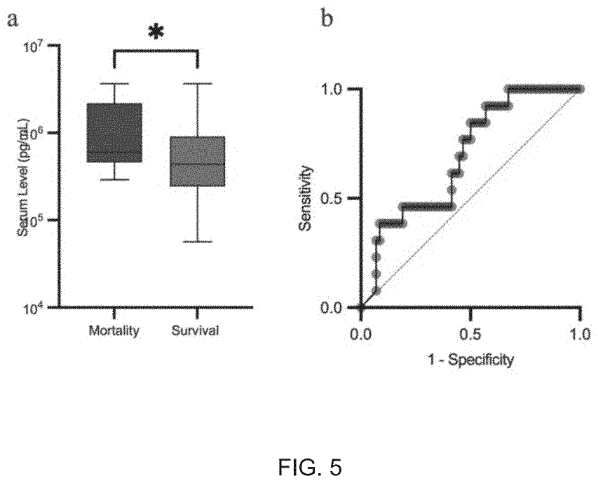

The study sought to determine if any of the PERSEVERE II biomarkers would perform better than PERSEVERE II to identify high-risk patients at the time of evaluation for sepsis. Amongst PERSEVERE II biomarkers in Cohort 1, IL-8 was the most statistically different between non-survivors and survivors, 1468 pg/mL vs. 219 pg/mL respectively, p 0.0001 (FIG. 3A). The area under the ROC curve for mortality was 0.83 (95% CI 0.72 to 0.94), p 0.0001 (FIG. 3B). A cutoff of 300 pg/mL was 92% sensitive and 62% specific for mortality (Table 2). Other markers were also statistically different between non-survivors and survivors, CCL3 was higher in non-survivors with a median of 81 pg/mL vs. a median of 45 pg/mL in survivors, p 0.009. the area under the ROC curve for mortality was 0.73 (95% CI 0.58 to 0.87), p 0.01 (FIG. 4). HSP A1b was also different between non-survivors and survivors but had a lower predictive capacity; 600765 pg/mL vs. 436901 pg/mL, p 0.03 with an AUC of 0.69 (0.54 to 0.83) (FIG. 5).

### Example 5

### nPERSEVERE-1: A Decision Tree That is Specific for Neonates

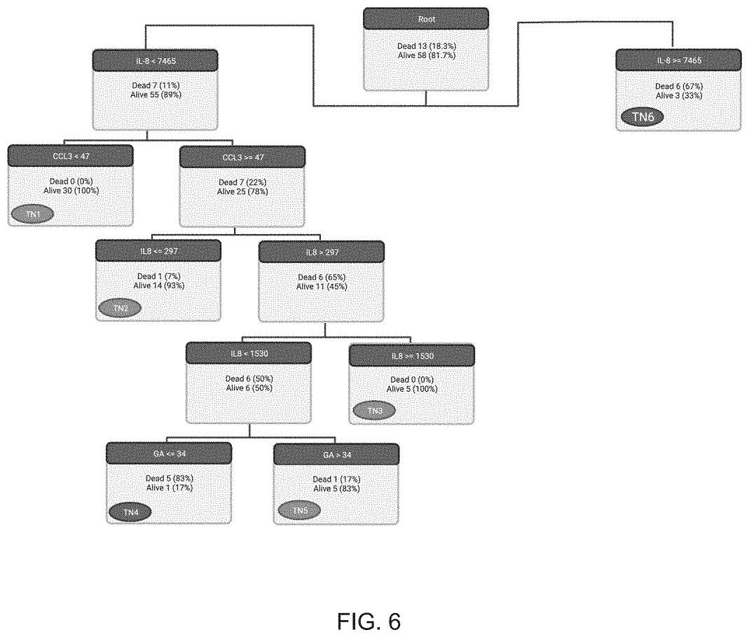

Since IL-8 showed good performance for mortality prediction in neonatal sepsis, the study tested if a new decision tree that is neonate-specific can perform better than IL-8 in Cohort 1. Using classification tree methodology, a new neonatal tree, nPERSEVERE-1, was derived that utilizes IL-8, CCL3, and gestational age to classify neonates according to mortality (FIG. 6). One of downfalls of classification trees is overfitting. To overcome this issue, both the gestational age and IL-8>=1530 pg/mL was pruned, and the performance of this pruned tree (FIG. 7) was assessed in Cohort 1. Neonates who were classified to terminal nodes one and two (predicted survivors) had a mortality rate of 2.2% compared to those who were classified to nodes three and four (predicted non-survivors) with a rate of 46.1%. The new pruned tree was 92% sensitive and 76% specific for mortality, p<0.0001. The area under the ROC curve for mortality was 0.89 (95% CI 0.81 to 0.97), p<0.0001. Due to the limited sample size, the new tree could not be tested in the clinical setting, but upon 10-fold cross validation the AUC was 0.73. Furthermore, neonates who were classified to terminal nodes one and two had a complicated course rate of 9.8% compared to 63.3% in terminal nodes three and four, p<0.0001.

Since there were patients in Cohort 1 that were classified as predicted non- survivors and did survive, the study investigated if they indeed had a higher baseline mortality risk, but the clinical care provided to them mitigated that outcome. Those who were predicted non-survivors and survived (n=14) were compared to those who were predicted survivors and survived (n=44). Predicted non-survivors had a higher trend that was nearing statistical significance in needing vasopressor support compared to predicted survivors, 42.9% vs. 15.9%, relative risk 2.7 (95% CI 1.1-6.4), and they spent more days on vasopressor support, 0 days (IQR 0-3.5) vs. 0 days (IQR 0-0) respectively, p 0.039. Furthermore, predicted non-survivors were more likely to have complicated course (two or more organ dysfunction on day 7 of illness) than predicted survivors, 35.7% vs. 11.4% respectively, relative risk 3.1 (95% CI 1.1-8.7), p 0.035.

### Example 6

### Survival Curves of High- and Low-Risk Patients According to PERSEVERE II and nPERSEVERE-1

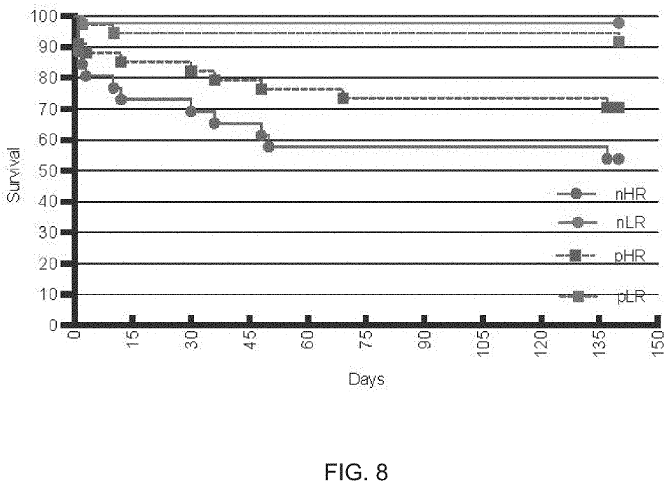

The survival curves of those who were classified as low risk and high risk were generated according to PERSEVERE and nPERSEVERE-1. Comparison of survival curves using Mantel-Cox test showed that the curves of high- and low-risk groups were statistically different, p 0.03 for PERSEVERE II and <0.0001 for nPERSEVERE-1 (FIG. 8).

### Example 7

### Cohort 2 Demographics

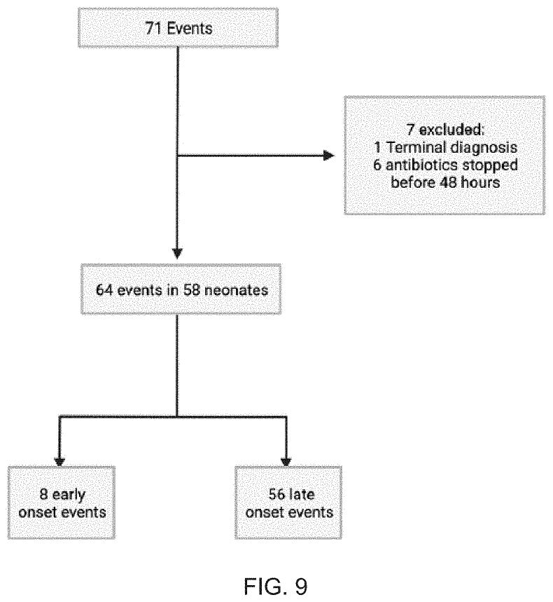

In total, 71 events were evaluated for enrollment in Cohort 2. 1 neonate was excluded for terminal diagnosis, and 6 neonates were excluded for discontinuation of antibiotics within 48 hours of evaluation. 58 neonates were included in the analysis who had a total of 64 events. Four neonates who had multiple sepsis events during their NICU stay (n=4), and only the last event they had was included in this analysis (FIG. 9). The overall mortality rate was 15.3%.

First, the median values of PERSEVERE biomarkers were compared between neonates with sepsis and neonates without inflammation (n=13). All the biomarkers were higher in neonates with sepsis, but only IL-8 and MMP-8 reached statistical significance (Table 3). Looking at neonates with sepsis, survivors and non-survivors were comparable in their characteristics. Although there was no statistical difference between both groups in all variables, including culture positivity rate, there was a trend for smaller birth weights, earlier gestational ages, and earlier late onset sepsis in non-survivors (Table 4). Further details regarding culture results can be found in Table 5.

TABLE 3
PERSEVERE Biomarkers Values in Controls and Neonates with Sepsis in Cohort
2. Data are presented as median and interquartile ranges. The unit is pg/mL
for all the serum levels. p value calculated using the Mann-Whitney test.

| PERSEVERE | Controls | | Sepsis | | |
|---|---|---|---|---|---|
| Biomarker | (n = 13) | | (n = 58) | | p Value |
| Granzyme B | 25.6 | [6.9-68.8] | 30.9 | [10.3-86.4] | 0.57 |
| IL-8 | 75.4 | [37.1-321.3] | 1468 | [257.1-81355] | 0.006 |
| Heatshock Protein A1b | 264687 | [198156-1083880] | 527698 | [275055-1017362] | 0.23 |
| CCL-3 | 45.8 | [33.1-243.1] | 48.9 | [30.6-97.3] | 0.48 |
| MMP-8 | 12110 | [5654-44565] | 32592 | [11738-1081800] | 0.02 |

TABLE 4
Characteristics of Neonates in Cohort 2. Survivors
and non-survivors are comparable.

| | Mortality n = 9 | | Survival n = 49 | |
|---|---|---|---|---|
| Characteristic | (16%) | | (84%) | p-value |
| Sex: Female | 7 | (78%) | 24 (49%) | 0.15 ^{a} |
| n (%) | | | | |
| Male n (%) | 2 | (22%) | 25_(51%) | |
| Race: Black | 3 | (33%) | 16 (33%) | |
| Others | 1 | (11%) | 1 (2%) | >0.99 ^{b} |
| White | 5 | (56%) | 32 (65%) | |
| Birth Weight | 1030 | [605-2289] | 1638 [799-2925] | 0.29 ^{c} |
| gram [IQR] | | | | |
| GA at Birth | 26 | [25-35] | 34 [26-37] | 0.19 ^{c} |
| Weeks [IQR] | | | | |
| Congenital | 1 | (11%) | 20 (41%) | 0.14 ^{a} |
| Anomalies | | | | |
| Positive Culture | 7 | (78%) | 42 (86%) | 0.61 ^{a} |
| Gram Negative | 3/7 | (43%) | 18/42 (43%) | >0.99 ^{d} |
| Gram Positive | 3/7 | (43%) | 21/42 (50%) | >0.99 ^{d} |
| Coagulase | 1/7 | (17%) | 7/42 (17%) | >0.99 ^{d} |
| Negative | | | | |
| Staph | | | | |
| Fungal | 1/7 | (17%) | 1/42 (18%) | >0.26 ^{d} |
| Early Vs. Late | 3 | (33%) | 5 (10%) | 0.10 ^{a} |
| Chronological | 12 | [8-41] | 27 [13-70] | 0.10 ^{c} |
| Age at Event | | | | |
| Days [IQR] ^{e} | | | | |

^{a} Calculated using the Fisher's exact test
^{b} Calculated using the Fisher's exact test comparing neonates who are black vs. white
^{c} Calculated using the Mann-Whitney test
^{d} Calculated using the Fisher's exact test comparing each category to the rest of cases with positive cultures
^{e} Included only late sepsis events

TABLE 5
Culture Results of the Entire Cohort 2. No statistical difference
was found between survivors and non-survivors.

| | Mortality n = 9 | Survival n = 49 | |
|---|---|---|---|
| Culture Growth | (16%) | (84%) | |
| E. Coli | 3 (33%) | 6 | (12%) |
| Klebsiella Species | — | 5 | (10%) |
| Pseudomonas Aeruginosa | — | 4 | (8%) |
| Hemophilus Influenzae | — | 1 | (2%) |
| Acinetobacter Species | — | 1 | (2%) |
| Enterobacter Species | — | 1 | (2%) |
| Staph Aureus | — | 8 | (16%) |
| Coagulase-negative Staph | 1 (11%) | 7 | (14%) |
| Group B Streptococcus | 1 (11%) | 1 | (2%) |
| Group A Streptococcus | — | 1 | (2%) |
| Stenotrophomonas maltophilia | 1 (11%) | — | |
| Enterococcus faecalis | — | 3 | (6%) |
| Other gram positives | — | 2 | (4%) |
| Candida albicans | 1 (11%) | 1 | (2%) |
| Enterovirus | — | 1 | (2%) |
| No growth | 2 (22%) | 7 | (14%) |

### Example 8

### Performance of PERSEVERE II for Neonates with Sepsis in Cohort 2

Neonates in Cohort 2 were classified according to the PERSEVERE II decision tree as described previously (FIG. 2). Neonates who were classified to terminal nodes one, two, and eight were considered low risk (predicted survivors), and those classified to terminal nodes three, four, six, seven, and nine were considered high risk (predicted non-survivors). The entire Cohort 2 had a mortality rate of 15.3%, and those who were classified as low risk had a mortality rate of 9.4% while high risk patients had a rate of 23.1%. PERSEVERE II was 67% sensitive and 59% specific for mortality, p 0.27 (Table 6). High risk patients also had higher complicated course rate nearing statistical significance compared to low-risk ones, 42.3% vs. 18.8% respectively, p 0.08.

### Example 9

### IL-8 is a Candidate Marker for Mortality Prediction in Cohort 2

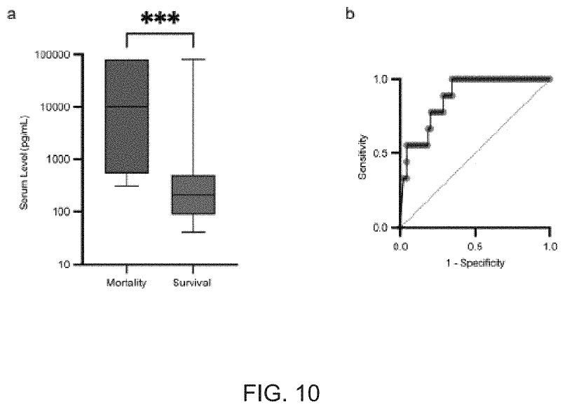

The study sought to determine if any of PERSEVERE II biomarkers alone would perform better than PERSEVERE II to identify high-risk patients at the time of evaluation for sepsis. Amongst PERSEVERE II biomarkers, IL-8 was the most statistically different between non-survivors and survivors in Cohort 2, with a median of 10 114 pg/mL [IQR 531-81 355 pg/mL] vs. 207 pg/mL [IQR 89-494 pg/mL] respectively, p 0.0001 (FIG. 10A). The area under the ROC curve for mortality was 0.88 (95% CI 0.77 to 0.98), p 0.0004 (FIG. 10B). Based on ROC calculations, a cutoff of 300 pg/mL had the highest specificity (65%) for mortality in Cohort 2, while retaining a 100% sensitivity, p 0.0003 (Table 6).

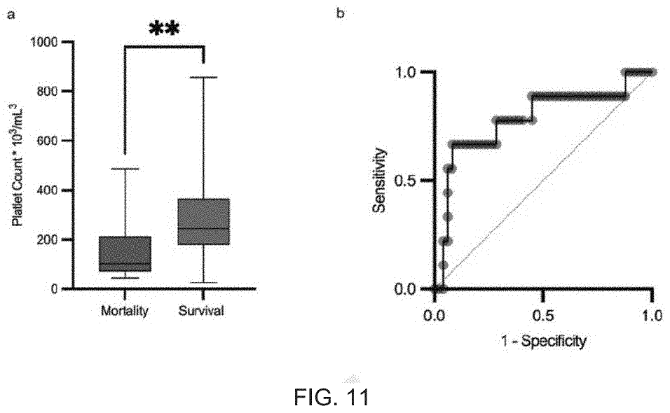

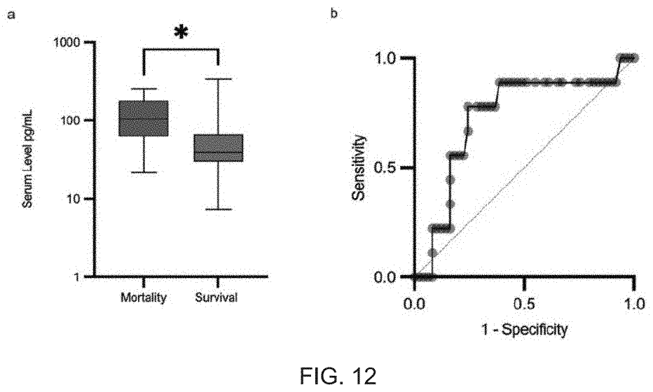

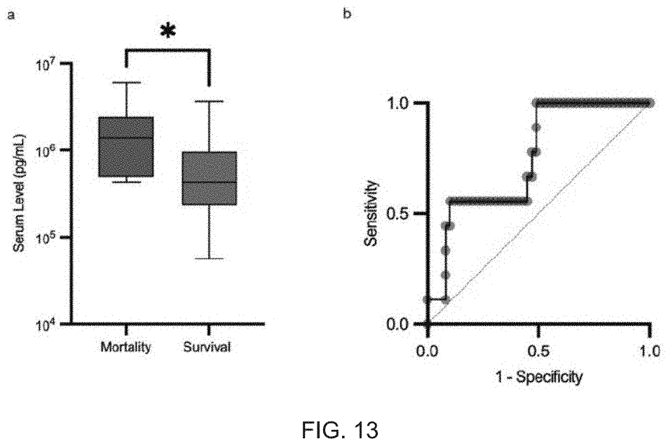

The next most statistically different biomarker between survivors and non-survivors was the platelet count. Non-survivors had a median count of 104 000 per mm³ [71 000-213 000 per mm³] compared to 246 000 per mm³ [180 000-365 000 per mm³] in survivors, p 0.006 (FIG. 11). Other markers were also statistically different between non-survivors and survivors, CCL3 was higher in non-survivors compared to survivors, 104 pg/mL [IQR 62-181 pg/mL] vs. 39 pg/mL [30-67 pg/mL] respectively, p 0.03 (FIG. 12). HSP A1b was also different between non-survivors and survivors and had predictive mortality capacity comparable to CCL-3; 1 390 000 pg/mL [IQR 497 047-2 415 000 pg/mL] vs.433 874 pg/mL [IQR 232 115-963 855 pg/mL] respectively, p 0.02 (FIG. 13). GZMB and MMP8 were not statistically different between survivors and non-survivors.

TABLE 6
Performance of Candidate Predictors of Mortality in Neonatal Sepsis in Cohort
2. Summary of candidate predictors of mortality. IL-8 is superior to PERSEVERE
II with improved sensitivity, specificity, negative predictive value, and
area under the curve. nPERSEVERE-2 is superior to IL- 8 with improved specificity,
+likelihood ratio, and area under the curve.

| Characteristic | PERSEVERE | IL-8 | nPERSEVERE-2 |
|---|---|---|---|
| Sensitivity (95% CI) | 67% (35-88%) | 100% (70-100%) | 100% (71-100%) |
| Specificity (95% CI) | 59% (45-72%) | 65% (51-77%) | 85% (73-93%) |
| NPV (95% CI) | 91% (76-97%) | 100% (89-100%) | 100% (92-100%) |
| PPV (95% CI) | 23% (11-42%) | 35% (19-54%) | 56% (33-77%) |
| +Likelihood Ratio | 1.6 | 2.9 | 7 |
| AUC (95% CI) | 0.65 (0.46-0.84) | 0.87 (0.77-0.98) | 0.95 (0.89-1.00) |
| The Fisher's Exact test p value | 0.27 | 0.0003 | <0.0001 |

### Example 10

### nPERSEVERE-2: A Decision Tree That is Specific for Neonates

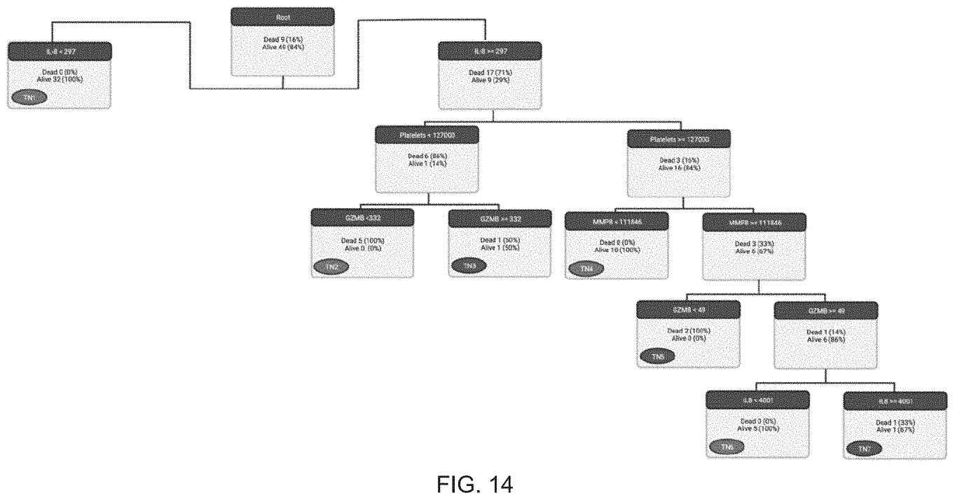

Since IL-8 showed good performance for mortality prediction in neonatal sepsis, the study tested if a new decision tree that is neonate-specific can perform better than IL-8. Using classification tree methodology, a new neonatal tree, nPERSEVERE-2, was rederived in Cohort 2, that utilizes IL-8, platelet count, CCL3, MMP8, and GZMB to classify neonates according to mortality (FIG. 14).

One of the main downfalls of classification trees is overfitting. To overcome this issue, the original tree was pruned by decreasing the number of branching and increasing the number of subjects in terminal nodes (FIG. 15). Neonates in Cohort 2 classified to terminal nodes one and three (predicted survivors) had a mortality rate of 0% and considered low risk. Meanwhile, neonates in Cohort 2 classified to nodes two and four (predicted non-survivors) had a mortality rate of 56% and considered high risk. The new pruned tree was 100% sensitive and 86% specific for mortality with a misclassification rate of 6%, p<0.0001. The area under the ROC curve for mortality was 0.95 (95% CI 0.89 to 1.00), p<0.0001.

To validate this model, 5-fold cross validation was performed. The average misclassification rate was 7%, and the calculated area under the curve showed good predictive capacity with an average of 0.86. Furthermore, neonates who were low risk had a complicated course rate of 14% compared to 69% in high risk neonates, p<0.0001.

Beyond 5-fold cross validation, when nPERSEVERE-2 was applied to the 6 events that were not included in the derivation cohort (prior multiple events), the model showed good performance with a misclassification rate of 15% and an area under the curve for all the events (n=65) was 0.88 (95% CI 0.77-0.99), p<0.0001. Also, there was a non-statistically significant trend of higher vasopressor use in predicted non-survivors compared to predicted survivors, 35% vs 24% respectively, p 0.25.

Since there were patients in Cohort 2 that were classified as predicted non-survivors and did survive, the study evaluated if they indeed had a higher baseline mortality risk, but the clinical care provided to them mitigated that outcome. Those who were predicted non-survivors and survived (n=7) were compared to those who were predicted survivors and survived (n=42). Predicted non-survivors were more likely to experience a complicated course of illness than predicted survivors, 29% vs. 22% respectively, but this difference was not significant, p 0.25. Furthermore, neonates who were predicted non-survivors had more organs injured compared to predicted survivors, 2 [IQR 1-3] vs. 1 [IQR 0-2], p 0.06. Amongst survivors, the use of vasopressor was comparable between the predicted survivors and predicted non-survivors, 28% vs. 24%, p>0.99.

### Example 11

### Survival Curves of High- and Low-Risk Patients According to nPERSEVERE-2

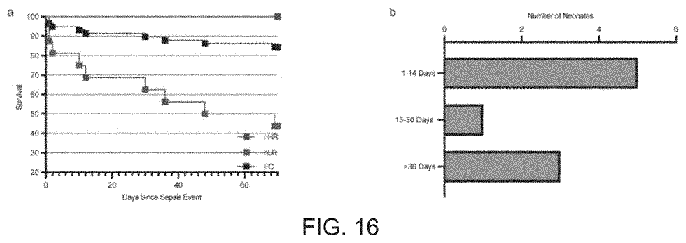

The survival curves of neonates in Cohort 2 classified as low risk and high risk were generated according to nPERSEVERE-2 to give a visual representation of the difference between the two groups. Comparison of survival curves using the Mantel-Cox test showed that the curves of high- and low-risk groups were statistically significant, p<0.0001 (FIG. 16A). Although most death events occurred early in the course (median day of death=12 days), some events ended up with late death after 30 days (3 events, 33% of total deaths) (FIG. 16B).

### Example 12

### nPERSEVERE-1 and nPERSEVERE-2 can Estimate Risk of Mortality and/or Complicated Course in Neonates

The studies described herein demonstrate the feasibility of stratification in neonates with sepsis at the time of evaluation according to their baseline mortality risk. These studies demonstrate the feasibility of stratification in neonates with sepsis at the time of evaluation according to their baseline mortality risk. First, PERSEVERE II was tested, showing that the cohort could be dichotomized into two groups with higher and lower mortality rates. PERSEVERE II was 77% sensitive and 59% specific for mortality in Cohort 1 and was 67% sensitive and 59% specific for mortality in Cohort 2 compared to a sensitivity of 86% and specificity of 69% in the pediatric cohort [9]. It is not surprising that PERSEVERE II did not perform as well in a neonate only population compared to its prior performance in various pediatric cohorts, since neonates have a unique early response in sepsis that does not match older pediatric age groups [17].

Second, using PERSEVERE biomarkers, this study demonstrated that IL-8 can be used as a single biomarker for mortality prediction in neonatal sepsis. IL-8 is a potent neutrophil chemoattractant and has been shown to be elevated in sepsis and septic shock and correlates with other pro-inflammatory cytokines such as IL-6 [18]. Higher levels of IL-8 can correlate with higher inflammatory burden in sepsis and potentially worse outcomes. Also, a prospective cohort study of adults with sepsis demonstrated that a cutoff IL-8 level of 94 pg/mL was 66% sensitive and 61% specific for mortality with a negative predictive value of 77%, p<0.0001 [19].

Finally, using classification tree methodology, the new decision trees, nPERSEVERE-1 and nPERSEVERE-2, were devised that performed very well in this cohort of neonates from two centers. nPERSEVERE-1 is 92% sensitive and has a negative predictive value of 97% for mortality with AUC of 0.89. nPERSEVERE-2 is 100% sensitive and has a negative predictive value of 100% for mortality with AUC of 0.95. Beyond the dichotomy of survival and death, it was demonstrated that those who were predicted non-survivors and did survive had a higher disease burden as evident by the higher need for vasopressor support during their illness and more organ dysfunctions at day 7 of illness and higher rate of complicated course.

### Example 13

### Use of nPERSEVERE-1 and nPERSEVERE-2 for Prognostication in Neonates with Sepsis

Biomarker research in neonatal sepsis has largely been focused on identifying a marker that can diagnose culture positive sepsis early in the course, but this approach has not yet translated to meaningful change in clinical practice. This study focused on the utility of prognostication in biomarker research, and there are many applications for nPERSEVERE-1 and nPERSEVERE-2. These tools can provide risk assessment at the time of evaluation rather than after certain time has lapsed since the diagnosis of sepsis, allowing for mortality risk allocation as soon as the neonate starts to show clinical evidence of the disease. This represents the first step towards precision medicine in neonatal sepsis and can provide meaningful data at the bedside. It alerts the clinical team very early in the course of the infection about those who are at high-risk of dying and can identify those who could benefit from high-risk therapies. This is also a helpful tool that aids in counseling families of neonates with sepsis, as it provides information about how sick they are from the time of sepsis evaluation.

Another meaningful and important use for nPERSEVERE-1 and nPERSEVERE-2 is in randomized clinical trials that ask whether certain interventions reduce mortality in neonates with sepsis. It allows for enrollment of neonates who have high baseline mortality risk, so that the intervention and placebo arms are equitable while also providing prognostic enrichment that reduces the number of subjects needed to answer the same question with equal power. Furthermore, these high-risk interventions, such as immunomodulation [20, 21] and higher dose antibiotic prescription [22], would be targeted towards patients who are more likely to see benefit from such interventions and would spare those who are at low risk of dying from potential side effects.

Furthermore, on a healthcare system level, nPERSEVERE-1 and nPERSEVERE-2 can serve as a valuable tool in quality improvement efforts that aims to reduce neonatal mortality from infections, as it not only provides a metric to gauge the effect of the efforts' interventions, but also focuses these efforts on those who have a high baseline risk of mortality. Also, this assay can guide clinicians to determine which neonates would benefit from transferring to a center with higher capabilities, as the assay focuses not on whether the patient has sepsis, but rather, if the patient will likely have organ failure or death.

The various methods and techniques described above provide a number of ways to carry out the invention. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the invention extend beyond the specifically disclosed embodiments to other alternative embodiments and/or uses and modifications and equivalents thereof.

In some embodiments, the numbers expressing quantities of ingredients, properties such as molecular weight, reaction conditions, and so forth, used to describe and claim certain embodiments of the application are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Preferred embodiments of this application are described herein. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the invention. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

### References

All of the publications mentioned herein, including those listed below, are incorporated by reference herein in their entirety.

1. Stoll, B. J., et al., Neonatal outcomes of extremely preterm infants from the NICHD Neonatal Research Network. Pediatrics, 2010. 126(3): p. 443-56.

2. Camacho-Gonzalez, A., P. W. Spearman, and B. J. Stoll, Neonatal infectious diseases: evaluation of neonatal sepsis. Pediatr Clin North Am, 2013. 60(2): p. 367-89.

3. Carr, R., N. Modi, and C. Dore, G-CSF and GM-CSF for treating or preventing neonatal infections. Cochrane Database Syst Rev, 2003(3): p. CD003066.

4. Ohlsson, A. and J. B. Lacy, Intravenous immunoglobulin for suspected or proven infection in neonates.Cochrane Database Syst Rev, 2020. 1: p. CD001239.

5. Brown, J. V. E., et al., Assessment of C-Reactive Protein Diagnostic Test Accuracy for Late-Onset Infection in Newborn Infants: A Systematic Review and Meta-analysis. JAMA Pediatr, 2020. 174(3): p. 260-268.

6. Ye, Q., Du, L. Z., Shao, W. X. & Shang, S. Q. Utility of cytokines to predict neonatal sepsis. Pediatr Res, 2017. 81, 616-621.

7. Zhou, M., et al., Interleukin-8 for diagnosis of neonatal sepsis: a meta-analysis. PLoS One, 2015. 10(5): p. e0127170.

8. Sherwin, C., et al., Utility of interleukin-12 and interleukin-10 in comparison with other cytokines and acute-phase reactants in the diagnosis of neonatal sepsis. Am J Perinatol, 2008. 25(10): p. 629-36.

9. Wong, H. R., et al., Prospective clinical testing and experimental validation of the Pediatric Sepsis Biomarker Risk Model. Sci Transl Med, 2019. 11(518).

10. Wong, H. R., et al., The pediatric sepsis biomarker risk model. Crit Care, 2012. 16(5): p. R174.

11. Wong, H. R., et al., Testing the prognostic accuracy of the updated pediatric sepsis biomarker risk model. PLoS One, 2014. 9(1): p. e86242.

12. Wong, H. R., et al., A multibiomarker-based outcome risk stratification model for adult septic shock*. Crit Care Med, 2014. 42(4): p. 781-9.

13. Jacobs, L., et al., The Pediatric Sepsis Biomarker Risk Model (PERSEVERE) Biomarkers Predict Clinical Deterioration and Mortality in Immunocompromised Children Evaluated for Infection. Sci Rep, 2019. 9(1): p. 424.

14. Harris, P. A. et al., Research electronic data capture (REDCap)—a metadata-driven methodology and workflow process for providing translational research informatics support. J Biomed Inform 42, 377-381 (2009).

15. Wynn, J. L. and H. R. Wong, Pathophysiology and treatment of septic shock in neonates. Clin Perinatol, 2010. 37(2): p. 439-79.

16. Jetton, J. G., et al., Incidence and outcomes of neonatal acute kidney injury (AWAKEN): a multicentre, multinational, observational cohort study. Lancet Child Adolesc Health, 2017. 1(3): p. 184-194.

17. Wynn, J. L., et al., The influence of developmental age on the early transcriptomic response of children with septic shock. Mol Med, 2011. 17(11-12): p. 1146-56.

18. Hack, C. E., et al., Interleukin-8 in sepsis: relation to shock and inflammatory mediators. Infect Immun, 1992. 60(7): p. 2835-42.

19. Anderson, B. J., et al., Plasma sTNFR1 and IL8 for prognostic enrichment in sepsis trials: a prospective cohort study. Crit Care, 2019. 23(1): p. 400.

20. Bao, Q., Lv, R. & Lei, M. IL-33 attenuates mortality by promoting IFN-gamma production in sepsis. Inflamm Res 67, 531-538 (2018).

21. Vanden Berghe, T. et al. Simultaneous targeting of IL-1 and IL-18 is required for protection against inflammatory and septic shock. Am J Respir Crit Care Med 189, 282291 (2014).

22. Lertwattanachai, T. et al. Clinical outcomes of empirical high-dose meropenem in critically ill patients with sepsis and septic shock: a randomized controlled trial. J Intensive Care 8, 26 (2020).

23. Singer, M., et al., The Third International Consensus Definitions for Sepsis and Septic Shock (Sepsis-3). JAMA, 2016. 315(8): p. 801-10.

24. Goldstein, B., et al., International pediatric sepsis consensus conference: definitions for sepsis and organ dysfunction in pediatrics. Pediatr Crit Care Med, 2005. 6(1): p. 2-8.

1. A method of classifying a neonate patient with sepsis as high risk of mortality and/or complicated course, or other than high risk of mortality and/or complicated course, the method comprising:
obtaining a serum sample from a neonate patient with sepsis at a first time point;
analyzing the sample to determine serum protein biomnarker concentrations of one or more biomarkers comprising IL-8,
determining whether the serum levels of each of the biomarkers are greater than a respective cut-off serum level; and
classifying the patient as high risk of mortality and/or complicated course, or other than high risk of mortality and/or complicated course, based on the determination of whether the serum levels of each of the biomarkers are greater than the respective cut-off serum level.

2. The method of claim 1, wherein a classification of other than high risk of mortality and/or complicated course comprises a non-elevated serum level of IL-8.

3. The method of claim 1 further comprising analyzing the sample to determine serum protein biomarker concentrations of one or more additional biomarkers comprising MMP8, and determining platelet count of the neonate patient, and wherein a classification of high risk of mortality and/or complicated course comprises:
a) an elevated serum level of IL-8, and a non-elevated median platelet count per mm³; or
b) an elevated serum level of IL-8, an elevated median platelet count per mm³, and an elevated serum level of MMP8;
and wherein a classification of other than high risk of mortality and/or complicated course comprises:
c) a non-elevated serum level of IL-8; or
d) an elevated serum level of IL-8, an elevated median platelet count per mm³, and a non-elevated serum level of MMP8.

4. The method of claim 1, further comprising analyzing the sample to determine serum protein biomarker concentrations of one or more additional biomarkers comprising CCL3, and wherein a classification of high risk of mortality and/or complicated course comprises:
a) a highly elevated level of IL-8; or
b) an elevated level of IL-8; and an elevated level of CCL3;
and wherein a classification of other than high risk of mortality and/or complicated course comprises:
c) a non-highly elevated level of IL-8, and a non-elevated level of CCL3; or
d) a non-elevated level of IL-8, and an elevated level of CCL3.

5.-6. (canceled)

7. The method of claim 1, wherein an elevated level of IL-8 corresponds to a serum IL-8 concentration greater than 297 pg/mL.

8. The method of claim 1, wherein the serum biomarker levels are determined by serum protein biomarker concentration and wherein the median platelet count is determined by counting the median number of platelets per mm³, and wherein:
a) an elevated level of IL-8 corresponds to a serum IL-8 concentration greater than 297 pg/mL;
b) an elevated median platelet count per mm³ corresponds to a median platelet count per mm³ greater than 127,000 per mm³; and
c) an elevated level of MMP8 corresponds to a serum MMP8 concentration greater than 111,846 pg/mL: or
wherein the serum biomarker levels are determined by serum protein biomarker concentration, and wherein:
d) an elevated level of IL-8 corresponds to a serum IL-8 concentration greater than 297 pg/mL;
e) a highly elevated level of IL-8 corresponds to a serum IL-8 concentration greater than 7465 pg/mL; and
f) an elevated level of CCL3 corresponds to a serum CCL3 concentration greater than 47 pg/mL.

9.-11. (canceled)

12. The method of claim 1, wherein the determination of whether the levels of the one or more biomarkers are non-elevated above a cut-off level comprises applying the biomarker expression level data to a decision tree comprising the one or more bimarkers.

13. The method of claim 1, wherein the classification of other than high risk of mortality and/or complicated course comprises a classification of low risk of mortality and/or complicated course.

14. The method of claim 1, wherein the complicated course comprises cardiovascular, respiratory, renal, hepatic, hematologic, and/or neurologic dysfunction; and/or wherein the complicated course comprises persistence of two or more organ dysfunctions on day 7 of illness and/or vasopressor use; and/or wherein the complicated course comprises dysfunction in one or more organs selected from heart, lungs, kidneys, liver, blood, and brain.

15.-18. (canceled)

19. The method of claim 1, wherein the classification is combined with one or more patient demographic data and/or clinical characteristics and/or results from other tests or indicia of sepsis and/or one or more additional biomarkers, and/or wherein the classification is combined with one or more additional population-based risk scores.

20. The method of claim 19, wherein the one or more additional biomarkers is selected from wherein the biomarkers further comprise one or more selected from the group consisting of Heat shock protein 70 kDA (HSP70), HSPA1b (Heatshork Protein A1b), GZMB (Granzyme B), Interleukin-1α (IL-1a), and CCL3 (CC Chemokine Ligand 3); and/or wherein the patient demographic data and/or clinical characteristics and/or results from other tests or indicia of sepsis comprise at least one selected from the group consisting of the sepsis causative organism, the presence or absence or chronic disease, and/or the chronological age, gestational age at birth, birth weight, gender, race, and/or co-morbidities of the patient.

21.-23. (canceled)

24. The method of claim 1, wherein the sample is obtained within the first hour of presentation with sepsis, or wherein the sample is obtained within the first 24 hours of presentation with sepsis.

25. The method of claim 1, further comprising administering a treatment comprising one or more high risk therapy to a neonate patient that is classified as high risk, or administering a treatment excluding a high risk therapy to a neonate patient that is not high risk, or to provide a method of treating a neonate patient with sepsis, thereby improving an outcome in the patient with sepsis.

26. The method of claim 25, wherein the one or more high risk therapy comprises at least one selected from the group consisting of immune enhancing and/or modulating therapy, high dose antibiotics, extracorporeal membrane oxygenation/life support, plasmapheresis, pulmonary artery catheterization, and/or high-volume continuous hemofiltration.

27. (canceled)

28. The method of claim 1, wherein the patient classified as high risk of mortality and/or complicated course is enrolled in a clinical trial.

29. (canceled)

30. The method of claim 25, further comprising:
obtaining a second sample from the treated patient at a second time point;
analyzing the second sample to determine the expression levels of expression levels of one or more biomarkers comprising IL-8;
determining whether the protein biomarker expression levels of each of the biomarkers are greater than a respective cut-off protein biomarker expression level;
classifying the patient as high risk of mortality and/or complicated course, or other than high risk of mortality and/or complicated course, based on the determination of whether the expression levels of each of the hiomarkers are greater than the respective cut-off expression level; and
maintaining the treatment being administered if the patient's high risk classification has not changed, or changing the treatment being administered if the patient's high risk classification has changed.

31. The method of claim 30, further comprising analyzing the second sample to determine the expression levels of expression levels of one or more biomarkers comprising MMPS8 and/or CCL3, and determining whether the protein hiomarker expression levels of each of the biomarkers are greater than a respective cut-off protein biomarker expression level; and further comprising determining platelet count of the neonate patient.

32.-36. (canceled)

37. The method of claim 30, wherein the patient classified as high risk after the second time point is administered one or more high risk therapy, or wherein the patient classified as high risk and administered one or more high risk therapy after the first time point is not classified as high risk after the second time point.

38.-42. (canceled)

43. The method of claim 1, wherein the patient additionally has necrotizing enterocolitis.

44. A diagnostic kit, test, or array comprising a reporter hybridization probe, and a capture hybridization probe specific for each of two or more mRNA, DNA, and/or protein biomarkers comprising IL-8 and further comprising MMP8 and/or CCL3.

45.-48. (canceled)
